(19) United States
(12) Patent Application Publication
(10) Pub. No.: US 2012/0178100 A1
(43) Pub. Date: Jul. 12, 2012

(54) Serum Markers Predicting Clinical Response to Anti-TNF Alpha Antibodies in Patients with Psoriatic Arthritis

(75) Inventors: Carrie Wagner, Chesterbrook, PA (US); Sudha Visvanathan, Hoboken, NJ (US)

(73) Assignee: CENTOCOR ORTHO BIOTECH INC., Horsham, PA (US)

(21) Appl. No.: 13/386,998

(22) PCT Filed: Jul. 12, 2010
(86) PCT No.: PCT/US10/41714

§ 371 Date: Jan. 25, 2012

Related U.S. Application Data
(60) Provisional application No. 61/228,994, filed on Jul. 28, 2009.

(51) Int. Cl. G01N 33/566; G01N 33/573; G06N 3/08; C12M 1/40

(52) U.S. Cl. 435/7.4; 435/7.92; 435/287.2; 706/20

(57) ABSTRACT

The invention provides tools for management of patients diagnosed with psoriatic arthritis, specifically, prior to the initiation of therapy with an anti-TNFα agent. The tools are specific markers and algorithms of predicting response to therapy based on standard clinical primary and secondary endpoints using serum marker concentrations. In one embodiment the baseline levels of VEGF, prostatic acid phosphatase, and adiponectin are used to predict the response at Week 14 after the initiation of therapy. In another embodiment, the change in a serum protein biomarker after 4 weeks of therapy is used such as MDC, lipoprotein a, and beta2-microglobulin.

### PRIORITY

The instant application claims priority to U.S. Provisional Application No. 61/228,994, which is incorporated by reference in its entirety.

### BACKGROUND OF THE INVENTION

1. Field of the Invention

The present invention relates to methods and procedures for the use of serum biomarkers to predict the response of patients diagnosed with psoriatic arthritis to treatment with anti-tumor necrosis factor alpha (TNFα) biologic therapeutics.

2. Description of the Related Art

The treatment of patients with psoriatic arthritis (PsA) with biologic therapies such as golimumab (a human anti-human TNFα monoclonal antibody) presents a number of challenges. The effectiveness of treatment and clinical study design is impacted by the ability to predict the PsA patients who will respond and which PsA patients will lose response following treatment with golimumab. Surrogate markers or biomarkers may be useful in answering these questions.

Biomarkers are defined as “a characteristic that is objectively measured and evaluated as an indicator of normal biologic processes, pathogenic processes, or pharmacologic responses to a therapeutic intervention.” Biomarker Working Group, 2001. Clin. Pharm. and Therap. 69: 89-95. The definition of a biomarker has recently been further defined as proteins in which the change of expression may correlate with an increased risk of disease or progression, or which may be predictive of a response to a given treatment.

Neutralization of TNFα through the addition of an anti-TNFα antibody or biologic to in vitro or in vivo systems, can modify the expression of inflammatory cytokines and a number of other serum protein and non-protein components. An anti-TNFα antibody added to cultured synovial fibroblasts reduced the expression of the cytokines IL-1, IL-6, IL-8, and GM-CSF (Feldmann & Maini (2001) Annu Rev Immunol 19:163-196). Rheumatoid arthritis (RA) patients who were treated with infliximab had decreased serum levels of TNFR1, TNFR2, IL-1R antagonist, IL-6, serum amyloid A, haptoglobin, and fibrinogen (Charles 1999 J Immunol 163:1521-1528). Other studies have shown that RA patients who are treated with infliximab had decreased serum levels of soluble(s) ICAM-3 and sP-selectin (Gonzalez-Gay, 2006 Clin Exp Rheumatol 24: 373-379), as well as a reduction in the levels of the cytokine IL-18 (Pittoni, 2002 Ann Rheum Dis 61:723-725; van Oosterhout, 2005 Ann Rheum Dis 64:537-543).

Elevated levels of C-reactive protein (CRP) have been observed in patients with various immune-mediated inflammatory diseases. These observations indicate that CRP may have potential value as a marker for anti-TNFα treatment. St Clair, 2004 Arthritis Rheum 50:3432-3443, showed that infliximab returned CRP to normal levels in patients with early RA. In refractory psoriatic arthritis (Feletar, 2004 Ann Rheum Dis 63:156-161), treatment with infliximab also returned CRP to normal levels. CRP levels have also been shown to be associated with joint damage progression in early RA patients treated only with methotrexate (Smolen, 2006 Arthritis Rheum 54:702-710). When infliximab treatment was added to the methotrexate treatment, the CRP levels were no longer associated with the progression of joint damage.

Strunk demonstrated that infliximab treatment in RA patients reduced the expression of inflammation-related cytokines such as IL-6, as well as angiogenesis related cytokines such as VEGF (vascular endothelial growth factor) (2006 Rheumatol Int. 26:252-256). Ulfgren (2000 Arthritis Rheum 43:2391-2396) showed that infliximab treatment reduced the synthesis of TNF, IL-1, and IL-1beta in the synovium within 2 weeks of treatment. Mastroianni (2005 Br J Dermatol 153:531-536) showed that reductions in VEGF, FGF, and MMP-2 were associated with significant improvement in the area and severity of psoriasis following treatment with infliximab. Visvanathan (Ann Rheum Dis 2008,67:511-517;) showed that infliximab treatment reduced the levels of IL-6, VEGF, and CRP in the serum of PsA patients, and that the reductions reflected improved disease activity measures. Adipocytokines, leptin, and adiponectin have identified roles in T-cell mediated inflammatory processes have also been recently been examined in relationship to RA and response to anti-TNF therapy (Popa, et al. 2009, J. Rheumatol. 35: 274-30).

Pre-treatment serum marker concentrations have also been associated with response to anti-TNFα treatment. A low baseline serum level of IL-2R was found to be associated with the clinical response to infliximab in patients with refractory RA (Kuuliala 2006). Visvanathan (2007a) showed that the treatment of RA patients with infliximab plus MTX induced a decrease in a number of inflammation-related markers, including MMP-3. The study data showed that baseline levels of MMP-3 correlated significantly with measures of clinical improvement one year post-treatment.

Few markers have been examined with specific reference to psoriatic arthritis. For example Fink (2007 Clin Experiment Rheum 25:305-308) compared VEGF in patients with active or inactive PsA and healthy controls noting that the levels were significantly higher in patients with active disease as compare to the other two groups and correlated with patients' clinical monitoring scores such as VAS and PASI.

Therefore, while a number of serum protein and non-protein markers of inflammation and systemic disease have been demonstrated to be modified during anti-TNFα treatment, a unique set of markers and a predictive algorithm have not, thus far, been discovered which is predictive of response or non-response for either all inflammatory diseases so treated or for specific diseases, such as psoriatic arthritis.

### SUMMARY OF THE INVENTION

The invention relates the use of multiple biomarkers to predict the response of a patient to treatment with anti-TNFα therapy, and more specifically, to determine if a patient will or will not respond to treatment. In addition, the invention can be used to determine if a patient has responded to treatment, and if the response will be sustained. In one aspect, the invention encompasses the use of a multi-component screen using patient serum samples to predict the response as well as non-response of patients with PsA to treatment with a TNFα neutralizing monoclonal antibody.

In one embodiment, specific marker sets identified in datasets from patients with PsA prior to the initiation of anti-TNFα therapy, having been correlated to actual clinical response assessment, are used to predict clinical response of PsA patients tested prior to treatment with anti-TNFα therapy. In a specific embodiment the marker set is two or more markers selected from the group consisting of adiponectin, MDC, PAP, SGOT, VEGF, lipoprotein A, and beta-2-microglobulin.

In another embodiment, specific marker sets identified in datasets from patients with PsA prior to and following the initiation of anti-TNFα therapy, having been correlated to actual clinical response assessment, are used to predict clinical response of PsA patients prior to treatment with anti-TNFα therapy. In a specific embodiment the marker set is two or more markers selected from the group consisting of adiponectin, MDC, PAP, SGOT, VEGF, lipoprotein A, and beta-2-microglobulin.

The invention also provides a computer-based system for predicting the response of a PsA patient to anti-TNFα therapy wherein the computer uses values from a patient's dataset to compare to a predictive algorithm, such as a decision tree, wherein the dataset includes the serum concentrations of one or more markers selected from the group consisting of adiponectin, MDC, PAP, SGOT, VEGF, lipoprotein A, and beta-2-microglobulin. In one embodiment, the computer-based system is a trained neural network for processing a patient dataset and produces an output wherein the dataset includes one or more serum marker concentrations selected from the group consisting of adiponectin, MDC, PAP, SGOT, VEGF, lipoprotein A, and beta-2-microglobulin.

The invention further provides a device capable of processing and detecting serum markers in a specimen or sample obtained from an PsA patient wherein the serum marker concentrations selected from the group consisting of adiponectin, MDC, PAP, SGOT, VEGF, lipoprotein A, and beta-2-microglobulin. In one embodiment, the device compares the information produced by detection of one of adiponectin, MDC, PAP, SGOT, VEGF, lipoprotein A, and beta-2-microglobul into an algorithm for predicting response or non-response to anti-TNFα therapy.

The invention also provides a kit comprising a device capable of processing and/or detecting serum markers in a specimen or sample obtained from an PsA patient wherein the serum marker concentrations selected from the group consisting of adiponectin, MDC, PAP, SGOT, VEGF, lipoprotein A, and beta-2-microglobulin whereby the processed and/or detected serum marker level may be compared to an algorithm for predicting response or non-response to anti-TNFα therapy.

### BRIEF DESCRIPTION OF THE FIGURES

FIGS. 1-2 are PsA response prediction models shown in the form of a decision tree based on the use of serum biomarkers and correlated to patient clinical responses assessed by ACRS20. The non-responder or “No” node means subjects in that node are predicted by the model to be non-responders, while a “Yes” node means subjects in that node are predicted by the model to be responders. Within the node, the number of actual non-responders and the number of actual responders in that node are shown separated by a “/” symbol.

FIG. 1 is a predictive model developed from baseline (Week 0) marker data analyzed by multiplexed method from study patients receiving golimumab using the ACR20 at Week14, where the initial classifier for a non-responder is based on VEGF (cutoff value <8.08, log scale) and the secondary classifier for a responder is based on VEGF (a cutoff value >=8.08, log scale), a PAP a cutoff value >=−2.29, log scale), and a tertiary classifier which is adiponectin (a cutoff value >=1.35, log scale). A patient is also predicted to be a non-responder based on VEGF (cutoff value >=8.08, log scale) and PAP <−2.29 or VEGF (cutoff value >=8.08, log scale), PAP >=−2.29 and adiponectin (cutoff value <1.35, log scale).

FIG. 2 is a predictive model developed from the change from baseline (Week 0) to Week 4 in marker level data analyzed by multiplexed method from study patients receiving golimumab and in ACR20 at Week14 where the initial responder criteria is the change in MDC (cutoff value >=−0.12, log scale) and the secondary classifier is the change in lipoprotein A (cutoff value <−0.23); when the change in lipoprotein A is greater than or equal to the cutoff value and the change in MDC is greater than or equal to the cutoff value, the patient is predicted to be a responder. Patients having a change in MDC <−0.12 are further classified based on the change in beta2-microglobulin (cutoff >=−0.11, log value) as responders and if the change in beta2-microglobulin is less than the cutoff value, as non-responders.

### DETAILED DESCRIPTION OF THE INVENTION

### Abbreviations

ACR, American College of Rheumatology score

CART, classification and regression tree model

CRP, C-reactive protein

DAS28, Disease Activity Index Score using 28 joints

DIP, distal interphalangeal

EIA, Enzyme Immunoassay

ELISA, Enzyme Linked Immunoassay

G-CSF=granulocyte colony stimulating factor

HAQ, health assessment questionnaire

MAP, multi-analyte profile

MDC, Macrophage-Derived Chemokine

NAPSI, nail psoriasis severity index

PAP, prostatic acid phosphatase

PASI, psoriatic arthritis severity index

PsA, psoriatic arthritis

SELDI, Surface Enhanced Laser Desorption and Ionization

SAP, serum amyloid P component

SGOT

TNFα/TNFα, Tumor Necrosis Factor alpha

TNFR, Tumor Necrosis Factor receptor

VEGF, Vascular Endothelial Growth Factor

IL, Interleukin

IL-1R, IL-1 receptor

VAS, visual analog score

### DEFINITIONS

A “biomarker” is defined as ‘a characteristic that is objectively measured and evaluated as an objective indicator of normal biological processes, pathogenic processes, or pharmacologic responses to a therapeutic intervention’ by the Biomarkers Definitions Working Group (Atkinson et al. 2001 Clin Pharm Therap 69(3):89-95). Thus, an anatomic or physiologic process can serve as a biomarker, for example, range of motion, as can levels of proteins, gene expression (mRNA), small molecules, metabolites or minerals, provided there is a validated link between the biomarker and a relevant physiologic, toxicologic, pharmacologic, or clinical outcome.

By “serum level” of a marker is meant the concentration of the marker measured by one or more methods, such as an immunoassay, typically ex vivo on a sample prepared from a specimen such as blood. The immunoassay uses immunospecific reagents, typically antibodies, for each marker and the assay may be performed in a variety of formats including enzyme-coupled reactions, e.g., EIA, ELISA, RIA, or other direct or indirect probe. Other methods of quantifying the marker in the sample such as electrochemical, fluorescence probe-linked detection, are also possible. The assay may also be “multiplexed” wherein multiple markers are detected and quantitated during a single sample interrogation.

Observational studies usually report their results as odds ratios (OR) or relative risks. Both are measures of the size of an association between an exposure (e.g., smoking, use of a medication, etc.) and a disease or death. A relative risk of 1.0 indicates that the exposure does not change the risk of disease. A relative risk of 1.75 indicates that patients with the exposure are 1.75 times more likely to develop the disease or have a 75 percent higher risk of disease. A relative risk of less than 1 indicates that the exposure decreases risk. Odds ratios are a way to estimate relative risks in case-control studies, when the relative risks cannot be calculated specifically. Although it is accurate when the disease is rare, the approximation is not as reliable when the disease is common.

Predictive values help interpret the results of tests in the clinical setting. The diagnostic value of a procedure is defined by its sensitivity, specificity, predictive value and efficiency. Any test method will produce True Positive (TP), False Negative (FN), False Positive (FP), and True Negative (TN). The “sensitivity” of a test is the percentage of all patients with disease present or that do respond who have a positive test or (TP/TP+FN)×100%. The “specificity” of a test is the percentage of all patients without disease or who do not respond, who have a negative test or (TN/FP+TN)×100%. The “predictive value” or “PV” of a test is a measure (%) of the times that the value (positive or negative) is the true value, i.e., the percent of all positive tests that are true positives is the Positive Predictive Value (PV+) or (TP/TP+FP)×100%. The “negative predictive value” (PV−) is the percentage of patients with a negative test who will not respond or (TN/FN+TN)×100%. The “accuracy” or “efficiency” of a test is the percentage of the times that the test give the correct answer compared to the total number of tests or (TP+TN/TP+TN+FP+FN)×100%. The “error rate” calculates from those patients predicted to respond who did not and those patients who responded that were not predicted to respond or (FP+FN/TP+TN+FP+FN)×100%. The overall test “specificity” is a measure of the accuracy of the sensitivity and specificity of a test do not change as the overall likelihood of disease changes in a population, the predictive value does change. The PV changes with a physician's clinical assessment of the presence or absence of disease or presence or absence of clinical response in a given patient.

A “decreased level” or “lower level” of a biomarker refers to a level that is quantifiably less than a predetermined value called the “cutoff value” and above the lower limit of quantitation (LLOQ). This determined “cutoff value” is specific for the algorithm and parameters related to patient sampling and treatment conditions.

A “higher level” or “elevated level” of a biomarker refers to a level that is quantifiably elevated relative to a predetermined value called the “cutoff value.” This “cutoff value” is specific for the algorithm and parameters related to patient sampling and treatment conditions.

The term “human TNFα” (abbreviated herein as hTNFα or simply TNF), as used herein, is intended to refer to a human cytokine that exists as a 17 kD secreted form and a 26 kD membrane associated form, the biologically active form of which is composed of a trimer of noncovalently bound 17 kD molecules. The term human TNFα is intended to include recombinant human TNFα (rhTNFα), which can be prepared by standard recombinant expression methods or purchased commercially (R & D Systems, Catalog No. 210-TA, Minneapolis, Minn.).

By “anti-TNFα” or simply “anti-TNF” therapy or treatment is meant the administration of a biologic molecule (biopharmaceutical) to a patient, capable of blocking, inhibiting, neutralizing, preventing receptor binding, or preventing TNFR activation by TNFα. Examples of such biopharmaceuticals are neutralizing MAbs to TNFα including but not limited those antibodies sold under the generic names of infliximab, adalimumab, and golimumab, and antibodies in clinical development. Also included are non-antibody constructs capable of binding TNFα such as the TNFR-immunoglobulin chimera known as Etanercept. The term includes each of the anti-TNFα human antibodies and antibody portions described herein as well as those described in U.S. Pat. Nos. 6,090,382; 6,258,562; 6,509,015, and in U.S. patent application Ser. Nos. 09/801,185 and 10/302,356. In one embodiment, the TNFα inhibitor used in the invention is an anti-TNFα antibody, or a fragment thereof, including infliximab (Remicade®, Johnson and Johnson; described in U.S. Pat. No. 5,656,272, incorporated by reference herein), CDP571 (a humanized monoclonal anti-TNF-alpha IgG4 antibody), CDP 870 (a humanized monoclonal anti-TNF-alpha antibody fragment), an anti-TNF dAb (Peptech), CNTO 148 (golimumab, WO 02/12502 and U.S. Pat. No. 7,250,165), and adalimumab (Humira® Abbott Laboratories, a human anti-TNF mAb, described in U.S. Pat. No. 6,090,382 as D2E7). Additional TNF antibodies which may be used in the invention are described in U.S. Pat. Nos. 6,593,458; 6,498,237; 6,451,983; and 6,448,380, each of which is incorporated by reference herein. In another embodiment, the TNFα inhibitor is a TNF fusion protein, e.g., etanercept (Enbrel®, Amgen; described in WO 91/03553 and WO 09/406,476, incorporated by reference herein). In another embodiment, the TNFα inhibitor is a recombinant TNF binding protein (r-TBP-I) (Serono).

By “sample” or “patient's sample” is meant a specimen which is a cell, tissue, or fluid or portion thereof extracted, produced, collected, or otherwise obtained from a patient suspected to having or having presented with symptoms associated with a TNFα-related disease.

### Overview

Recent advances in technologies such as proteomics present pathologists with the challenge of integrating the new information generated with high-throughput methods with current diagnostic models based on clinicopathologic correlations and often with the inclusion of histopathological findings. Parallel developments in the field of medical informatics and bioinformatics provide the technical and mathematical methods to approach these problems in a rational manner providing new tools to the practitioner and pathologist or other medical specialists in the form multivariate and multidisciplinary diagnostic and prognostic models that are hoped to provide more accurate, individualized patient-based information. Evidence-based medicine (EBM) and medical decision analysis (MDA) are among the disciplines that use quantitative methods to assess the value of information and integrate so-called best evidence into multivariate models for the assessment of prognosis, response to therapy, and selection of laboratory tests that can influence individual patient care.

The subject matter disclosed and claimed herein includes several aspects such as:
1. The use of serum or other sample types to identify biomarkers associated with the response or non-response to anti-TNF, such as golimumab, treatment in patients with PsA;
2. The ability to predict a response or non-response to an anti-TNFα Mab, such as golimumab, treatment using biomarkers present in serum or other sample types from a diagnosed PsA patient prior to initiating anti-TNF therapy;
3. An algorithm to predict outcome in patients with PsA treated with anti-TNF therapy;
   a. The clinical response or non-response of PsA patients to anti-TNFα at Week 14 or later visits may be predicted at the time of assessment (Week 0) using biomarkers present in a diagnosed PsA patient's serum or other sample types prior to the initiation of anti-TNF therapy.
   b. The clinical response or non-response of PsA patients to anti-TNFα treatment at Week 14 or later visits may be predicted using the change in biomarkers from a baseline value obtained prior to the initiation of therapy (Week 0) and at Week 4 after initiation of therapy.
   c. The clinical response or non-response of PsA patients to anti-TNFα treatment at Week 14 or later visits may be predicted using the change in biomarkers from a baseline value obtained prior to the initiation of therapy (Week 0) in combination with the change in biomarkers at Week 4 after initiation of therapy; and
4. Devices, systems, and kits comprising means for using the markers of the invention to predict response or non-response of a PsA patient to anti-TNFα therapy.

In order to define the markers useful in developing a predictive algorithm based on the concentrations of markers, serum was obtained from patients who had been treated with golimumab. Serum can be obtained at baseline (Week 0), Week 4, and Week 14 of treatment or other intermediate or longer time points. A number of biomarkers in the serum samples are analyzed, and the baseline concentration as well as the change in the concentration of biomarkers after treatment is determined The baseline and change in biomarker expression is then used to determine if the biomarker expression correlates with the treatment outcome at Week 14 or other defined time point after the initiation of treatment as assessed by the ACR20 or another measure of clinical response. In one embodiment, the process for defining the markers associated with the clinical response of a patient with PsA to anti-TNFα therapy and developing an algorithm for predicting response or non-response involving the serum concentrations of those markers uses a stepwise analysis wherein the initial correlations are done by logistic regression analysis relating the value for each biomarker for each patient at Week 0, 4, and 14 to the clinical assessment for that patient at Week 14 and 24 and once the ability of a marker to significantly correlate to response to therapy at multiple clinical endpoints is determined, a unique algorithm based on defined serum values of a marker or marker set is developed using CART or other suitable analytic method as described herein or known in the art.

In addition to the other markers disclosed herein, the dataset markers may be selected from one or more clinical indicia, examples of which are age, race, gender, blood pressure, height and weight, body mass index, CRP concentration, tobacco use, heart rate, fasting insulin concentration, fasting glucose concentration, diabetes status, use of other medications, and specific functional or behavioral assessments, and/or radiological or other image-based assessments wherein a numerical values are applied to individual measures or an overall numerical score is generated. Clinical variables will typically be assessed and the resulting data combined in an algorithm with the above described markers.

Prior to input into the analytical process, the data in each dataset is collected by measuring the values for each marker, usually in triplicate or in multiple triplicates. The data may be manipulated, for example, raw data may be transformed using standard curves, and the average of triplicate measurements used to calculate the average and standard deviation for each patient. These values may be transformed before being used in the models, e.g., log-transformed, Box-Cox transformed (see Box and Cox (1964) J. Royal Stat. Soc, Series B, 26:211-212; 1964), or other transformations known and practiced in the art. This data can then be input into the analytical process with defined parameters.

The quantitative data thus obtained related to the protein markers and other dataset components is then subjected to an analytic process with parameters previously determined using a learning algorithm, i.e., inputted into a predictive model, as in the examples provided herein (Examples 1-3). The parameters of the analytic process may be those disclosed herein or those derived using the guidelines described herein. Learning algorithms such as linear discriminant analysis, recursive feature elimination, a prediction analysis of microarray, logistic regression, CART, FlexTree, LART, random forest, MART, or another machine learning algorithm are applied to the appropriate reference or training data to determine the parameters for analytical processes suitable for a PsA response or non-response classification.

The analytic process may set a threshold for determining the probability that a sample belongs to a given class. The probability preferably is at least 50%, or at least 60% or at least 70% or at least 80% or higher.

In other embodiments, the analytic process determines whether a comparison between an obtained dataset and a reference dataset yields a statistically significant difference. If so, then the sample from which the dataset was obtained is classified as not belonging to the reference dataset class. Conversely, if such a comparison is not statistically significantly different from the reference dataset, then the sample from which the dataset was obtained is classified as belonging to the reference dataset class.

In general, the analytical process will be in the form of a model generated by a statistical analytical method such as a linear algorithm, a quadratic algorithm, a polynomial algorithm, a decision tree algorithm, a voting algorithm.

### Use of Reference/Training Datasets to Determine Parameters of Analytical Process

Using any suitable learning algorithm, an appropriate reference or training dataset is used to determine the parameters of the analytical process to be used for classification, i.e., develop a predictive model.

The reference, or training dataset, to be used will depend on the desired PsA classification to be determined, e.g., responder or non-responder. The dataset may include data from two, three, four, or more classes.

For example, to use a supervised learning algorithm to determine the parameters for an analytic process used to predict response to anti-TNFα therapy, a dataset comprising control and diseased samples is used as a training set. Alternatively, a supervised learning algorithm is to be used to develop a predictive model for PsA disease therapy.

### Statistical Analysis

The following are examples of the types of statistical analysis methods that are available to one of skill in the art to aid in the practice of the disclosed methods. The statistical analysis may be applied for one or both of two tasks. First, these and other statistical methods may be used to identify preferred subsets of the markers and other indicia that will form a preferred dataset. In addition, these and other statistical methods may be used to generate the analytical process that will be used with the dataset to generate the result. Several of statistical methods presented herein or otherwise available in the art will perform both of these tasks and yield a model that is suitable for use as an analytical process for the practice of the methods disclosed herein.

In a specific embodiment, biomarkers and their corresponding features (e.g., expression levels or serum levels) are used to develop an analytical process, or plurality of analytical processes, that discriminate between classes of patients, e.g., responder and non-responder to anti-TNFα therapy. Once an analytical process has been built using these exemplary data analysis algorithms or other techniques known in the art, the analytical process can be used to classify a test subject into one of the two or more phenotypic classes (e.g., a patient predicted to respond to anti-TNFα therapy or a patient who will not respond). This is accomplished by applying the analytical process to a marker profile obtained from the test subject. Such analytical processes, therefore, have value as diagnostic indicators.

In one aspect, the disclosed methods provide for the evaluation of a marker profile from a test subject to marker profiles obtained from a training population. In some embodiments, each marker profile obtained from subjects in the training population, as well as the test subject, comprises a feature for each of a plurality of different markers. In further embodiments, this comparison is accomplished by (i) developing an analytical process using the marker profiles from the training population and (ii) applying the analytical process to the marker profile from the test subject. As such, the analytical process applied in some embodiments of the methods disclosed herein is used to determine whether a test PsA patient is predicted to respond to anti-TNFα therapy or a patient who will not respond.

Thus, in some embodiments, the result in the above-described binary decision situation has four possible outcomes: (i) a true responder, where the analytical process indicates that the subject will be a responder to anti-TNFα therapy and the subject responds to anti-TNFα therapy during the definite time period (true positive, TP); (ii) false responder, where the analytical process indicates that the subject will be a responder to anti-TNFα therapy and the subject does not respond to anti-TNFα therapy during the definite time period (false positive, FP); (iii) true non-responder, where the analytical process indicates that the subject will not be a responder to anti-TNFα therapy and the subject does not respond to anti-TNFα therapy during the definite time period (true negative, TN); or (iv) false non-responder, where the analytical process indicates that the patient will not be a responder to anti-TNFα therapy and the subject does in fact respond to anti-TNFαtherapy during the definite time period (false negative, FN).

Relevant data analysis algorithms for developing an analytical process include, but are not limited to, discriminant analysis including linear, logistic, and more flexible discrimination techniques (see, e.g., Gnanadesikan, 1977, Methods for Statistical Data Analysis of Multivariate Observations, New York: Wiley 1977, which is hereby incorporated by reference herein in its entirety); tree-based algorithms such as classification and regression trees (CART) and variants (see, e.g., Breiman, 1984, Classification and Regression Trees, Belmont, Calif.; Wadsworth International Group); generalized additive models (see, e.g., Tibshirani, 1990, Generalized Additive Models, London: Chapman and Hall); and neural networks (see, e.g., Neal, 1996, Bayesian Learning for Neural Networks, New York: Springer-Verlag; and Insua, 1998, Feedforward neural networks for nonparametric regression In: Practical Nonparametric and Semiparametric Bayesian Statistics, pp. 181-194, New York: Springer. These references are hereby incorporated by reference in their entirety.

In a specific embodiment, a data analysis algorithm of the invention comprises Classification and Regression Tree (CART), Multiple Additive Regression Tree (MART), Prediction Analysis for Microarrays (PAM) or Random Forest analysis. Such algorithms classify complex spectra from biological materials, such as a blood sample, to distinguish subjects as normal or as possessing biomarker expression levels characteristic of a particular disease state. In other embodiments, a data analysis algorithm of the invention comprises ANOVA and nonparametric equivalents, linear discriminant analysis, logistic regression analysis, nearest neighbor classifier analysis, neural networks, principal component analysis, quadratic discriminant analysis, regression classifiers and support vector machines.

While such algorithms may be used to construct an analytical process and/or increase the speed and efficiency of the application of the analytical process and to avoid investigator bias, one of ordinary skill in the art will realize that a computer-based device is not required to carry out the methods of using the predictive models of the present invention.

### Results of the CART Analysis

In one aspect of the present invention, the analyses of serum markers in patients diagnosed with PsA was focused on significant relationships between biomarker baseline values and response to anti-TNFα therapy. In another aspect of the present invention, the analyses of the change in serum markers from baseline (prior to anti-TNFα therapy) to Week 4 after therapy in serum markers in patients diagnosed with PsA was related to the clinical response or non-response of the patient at a later time (Week 14).

In a specific embodiment of the invention, it was found that the baseline concentration of VEGF could be an initial classifier for predicting the Week 14 outcome assessed as ACR20 for the patients treated with golimumab. In an alternate embodiment, other baseline markers such as adiponectin, PAP and SGOT may be used as an initial classifier for predicting the Week 14 or Week 24 or outcome at other timepoints assessed as ACR20, DAS28, or PCS, PASI, or other methods of scoring active disease for the patients treated with golimumab. This information can be used by physicians to determine who is benefiting from golimumab treatment, and just as important, to identify those patients are not benefiting from such treatment.

Alternatively, DAS28 was used as the clinical outcome component of the model and VEGF at baseline, adiponectin at baseline, PAP at baseline, or SGOT at baseline or the change in was the initial marker for classification. Other baseline marker levels shown to be correlative to at least one Week 14 or Week 24 clinical response include IL-8, deoxypyridinoline, S-100 (acute phase proteins produced by monocytes and elevated in serum and SF from RA and PsA patients), hyaluronic acid, bone alkaline phosphatase, IL-6 (serum), and VEGF (serum).

Baseline Biomarkers Prediction of Response to anti-TNFα Therapy.

When a predictive algorithm was built from datasets comprising only the baseline biomarkers serum concentration values and correlated with clinical response of a PsA patient treated with an anti-TNFα therapeutic in more than one method of assessing clinical response, such as ACR20 and DAS28, the markers included VEGF, PAP, and adiponectin.

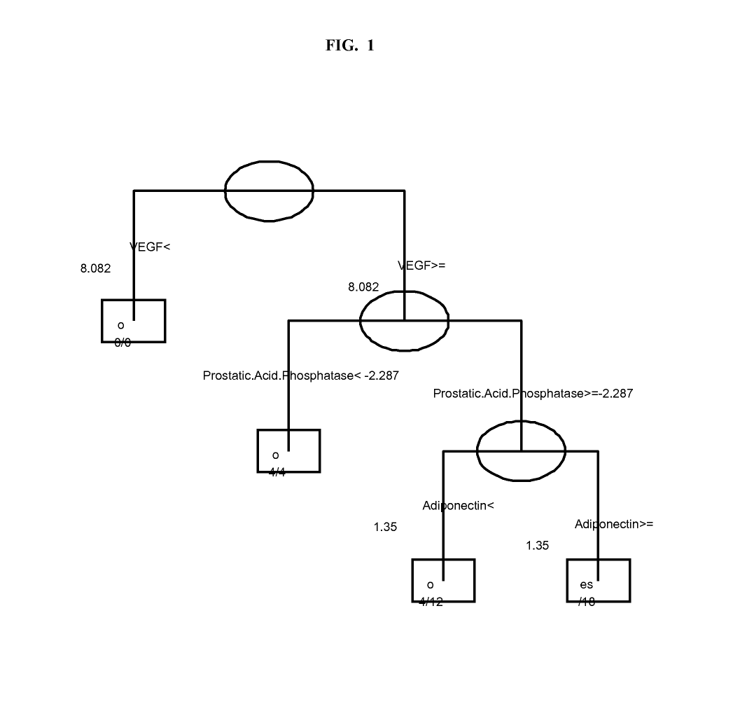

The CART model in FIG. 1 uses 3 markers to classify patients as responders or non-responders. For each marker, a single threshold is used (e.g., for VEGF, the threshold is 8.082). Patients are classified in such a model by using their biomarker values to proceed from the top of the decision tree to the bottom. Once a node at the bottom of the tree is reached, the classification for that patient is determined by the node label (either Yes or No to denote responders and non-responders, respectively). As an example, consider a patient with the following values:

VEGF=9.00

Prostatic Acid Phosphatase (PAP)=1.00

Adiponectin=1.00

At the top of the tree, the first marker is VEGF, and the threshold is 8.082. Since the VEGF value is 9.00 in this example, the right branch of the tree is followed. The next marker is PAP, the value 1.00 is greater than −2.287, so again the right branch is taken. Finally, the value of Adiponectin is 1.00, less than the threshold of 1.35, so the left branch is taken. The end result is the patient's values put them in a “No” bin, and the subject is classified as a non-responder. Note that in some cases, due to the hierarchical nature of the CART model, a patient may be classified on the basis of the top level marker only (e.g., if VEGF <8.082, the subject is classified as a non-responder regardless of the values of the other two markers in the model).

As demonstrated herein, analysis of biomarkers in serum obtained from PsA patients at baseline (Week 0, prior to treatment), quantitated by a multiplexed assay, the best CART model included VEGF as the initial classifier (FIG. 1) and PAP as the secondary classifier with adiponectin as a tertiary classifier when PAP was greater than or equal to a threshold level in patients having VEGF greater than or equal to a threshold level. The model sensitivity was 53%, and model specificity was 95%.

These results suggest that baseline levels of biomarkers can be measured prior to treatment by a physician to identify which of the patients treated with golimumab will respond or not respond to the treatment.

### Biomarker Change as Early Predictor of Outcome

When comparing the change in baseline serum levels at Week 4 in PsA patients, golimumab-treated patient groups demonstrated significantly different serum biomarker levels compared to the placebo-treated group. The biomarkers that changed included: alpha-1-Antitrypsin, CRP, ENRAGE, haptoglobin, ICAM-1, IL-16, IL-18, IL-1ra, IL-8, MCP-1, MIP-1beta, MMP-3, myeloperoxidase, serum amyloid P, thyroxine binding globulin, TNFRII, and VEGF.

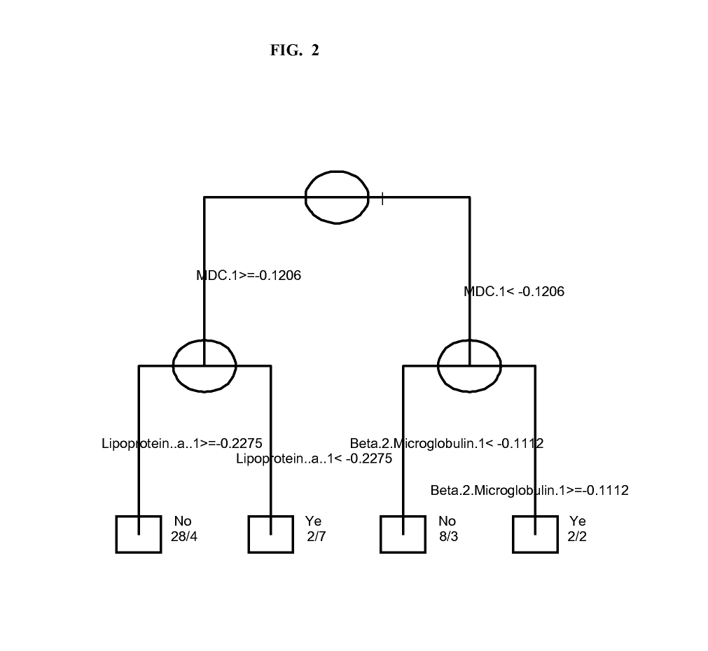

For analysis of biomarkers in serum obtained from PsA patients at baseline and Week 4 correlated to the primary clinical endpoint at Week 14 (ACR20), the biomarker model uses the change in MDC as the initial classifier followed by two subclassifications using change in lipoprotein A and in beta2-microglobulin (FIG. 2).

The specific examples described herein for generating an algorithm useful for predicting the response or non-response of a PsA patient to anti-TNFα therapy indicate that multiple markers are correlative of PsA processes and the quantitative interpretation of each particular biomarker in diagnosing or predicting response to therapy has not been heretofore well established. The applicants demonstrated that an algorithm can be generated using a sampling of patient data based on specific markers defined. In one method of using the markers of the invention, a computer assisted device is used to capture patient data and perform the necessary analysis. In another aspect, the computer assisted device or system may use the data presented herein as a “training data set” in order to generate the classifier information required to apply the predictive analysis.

### Instruments, Reagents and Kits for Performing the Analysis

The measurement of serum biomarkers for predicting response of a diagnosed PsA patient to anti-TNF therapy may be performed in a clinical or research laboratory or a centralized laboratory in a hospital or non-hospital location using standard immunochemical and biophysical methods as described herein. The marker quantitation may be performed at the same time as e.g., other standard measures such as WBC count, platelets, and ESR. The analysis may be performed individually or in batches using commercial kits, or using multiplexed analysis on individual patient samples.

In one aspect of the invention, individual and sets of reagents are used in one or more steps to determine relative or absolute amounts of a biomarker, or panel or biomarkers, in a patient's sample. The reagents may be used to capture the biomarker, such as an antibody immunospecific for a biomarker, which forms a ligand biomarker pair detectable by an indirect measurement such as enzyme-linked immunospecific assay. Either single analyte EIA or multiplexed analysis can be performed. Multiplexed analysis is a technique by which multiple, simultaneous EIA-based assays can be performed using a single serum sample. One platform useful to quantify large numbers of biomarkers in a very small sample volume is the xMAP® technology used by Rules Based Medicine in Austin, Tex. (owned by the Luminex Corporation), which performs up to 100 multiplexed, microsphere-based assays in a single reaction vessel by combining optical classification schemes, biochemical assays, flow cytometry and advanced digital signal processing hardware and software. In the technology, multiplexing is accomplished by assigning each analyte-specific assay a microsphere set labeled with a unique fluorescence signature. Multiplexed assays are analyzed in a flow device that interrogates each microsphere individually as it passes through a red and green laser. Alternatively, methods and reagents are used to process the sample for detection and possible quantitation using a direct physical measurement such as mass, charge, or a combination such as by SELDI. Quantitative mass spectrometric multiple reaction monitoring assays have also been developed such as those offered by NextGen Sciences (Ann Arbor, Mich.).

According to one aspect of the invention, therefore, the detection of biomarkers for evaluation of PsA status entails contacting a sample from a subject with a substrate, e.g., a probe, having capture reagent thereon, under conditions that allow binding between the biomarker and the reagent, and then detecting the biomarker bound to the adsorbent by a suitable method. One method for detecting the marker is gas phase ion spectrometry, for example, mass spectrometry. Other detection paradigms that can be employed to this end include optical methods, electrochemical methods (voltometry, amperometry or electrochemiluminescent techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry), and enzyme-coupled colorimetric or fluorescent methods.

Specimens from patients may require processing prior to applying the detecting method to the processed specimen or sample such as but not limited to methods to concentrate, purify, or separate the marker from other components of the specimen. For example a blood sample is typically allowed to clot followed by centrifugation to produce serum or treated with an anticoagulant and the cellular components and platelets removed prior to being subjected to methods of detecting analyte concentration. Alternatively, the detecting may be accomplished by a continuous processing system which may incorporate materials or reagents to accomplish such concentrating, separating or purifying steps. In one embodiment the processing system includes the use of a capture reagent. One type of capture reagent is a “chromatographic adsorbent,” which is a material typically used in chromatography. Chromatographic adsorbents include, for example, ion exchange materials, metal chelators, immobilized metal chelates, hydrophobic interaction adsorbents, hydrophilic interaction adsorbents, dyes, simple biomolecules (e.g., nucleotides, amino acids, simple sugars and fatty acids), mixed mode adsorbents (e.g., hydrophobic attraction/electrostatic repulsion adsorbents). A “biospecific” capture reagent is a capture reagent that is a biomolecule, e.g., a nucleotide, a nucleic acid molecule, an amino acid, a polypeptide, a polysaccharide, a lipid, a steroid or a conjugate of these (e.g., a glycoprotein, a lipoprotein, a glycolipid). In certain instances the biospecific adsorbent can be a macromolecular structure such as a multiprotein complex, a biological membrane or a virus. Illustrative biospecific adsorbents are antibodies, receptor proteins, and nucleic acids. A biospecific adsorbent typically has higher specificity for a target analyte than a chromatographic adsorbent.

The detection and quantitation of the biomarkers according to the invention can thus be enhanced by using certain selectivity conditions, e.g., adsorbents or washing solutions. A wash solution refers to an agent, typically a solution, which is used to affect or modify adsorption of an analyte to an adsorbent surface and/or to remove unbound materials from the surface. The elution characteristics of a wash solution can depend, for example, on pH, ionic strength, hydrophobicity, degree of chaotropism, detergent strength, and temperature.

In one aspect of the present invention, a sample is analyzed in a multiplexed manner meaning that the processing of markers from a patient samples occurs nearly simultaneously. In one aspect, the sample is contacted by a substrate comprising multiple capture reagents representing unique specificity. The capture reagents are commonly immunospecific antibodies or fragments thereof. The substrate may be a single component such as a “biochip,” a term that denotes a solid substrate, having a generally planar surface, to which a capture reagent(s) is attached, or the capture reagents may be segregated among a number of substrates, as for example bound to individual spherical substrates (beads). Frequently, the surface of a biochip comprises a plurality of addressable locations, each of which has the capture reagent bound there. A biochip can be adapted to engage a probe interface and, hence, function as a probe in gas phase ion spectrometry preferably mass spectrometry. Alternatively, a biochip of the invention can be mounted onto another substrate to form a probe that can be inserted into the spectrometer. In the case of the beads, the individual beads may be partitioned or sorted after exposure to the sample for detection.

A variety of biochips are available for the capture and detection of biomarkers, in accordance with the present invention, from commercial sources such as Ciphergen Biosystems (Fremont, Calif.), Perkin Elmer (Packard BioScience Company (Meriden Conn.), Zyomyx (Hayward, Calif.), and Phylos (Lexington, Mass.), GE Healthcare, Corp. (Sunnyvale, Calif.). Exemplary of these biochips are those described in U.S. Pat. No. 6,225,047, supra, and No. 6,329,209 (Wagner et al.), and in WO 99/51773 (Kuimelis and Wagner), WO 00/56934 (Englert et al.) and particularly those which use electrochemical and electrochemiluminescence methods of detecting the presence or amount of an analyte marker in a sample such as those multi-specific, multi-array taught in Wohlstadter et al., WO98/12539 and U.S. Pat. No. 6,066,448.

A substrate with biospecific capture and/or detection reagents is contacted with the sample, containing e.g., serum, for a period of time sufficient to allow the biomarker that may be present to bind to the reagent. In one embodiment of the invention, more than one type of substrate with biospecific capture or detection reagents thereon is contacted with the biological sample. After the incubation period, the substrate is washed to remove unbound material. Any suitable washing solutions can be used; preferably, aqueous solutions are employed.

Biomarkers bound to the substrates are to be detected after desorption directly by using a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined. Such methods may be used to discovery biomarkers and, in some instances for quantitation of biomarkers.

In another embodiment, the method of the invention is a microfluidic device capable of miniaturized liquid sample handling and analysis device for liquid phase analysis as taught in, for example, U.S. Pat. No. 5,571,410 and U.S. Pat. No. RE36350, useful for detecting and analyzing small and/or macromolecular solutes in the liquid phase, optionally, employing chromatographic separation means, electrophoretic separation means, electrochromatographic separation means, or combinations thereof. The microfluidic device or “microdevice” may comprise multiple channels arranged so that analyte fluid can be separated, such that biomarkers may be captured, and, optionally, detected at addressable locations within the device (U.S. Pat. No. 5,637,469, U.S. Pat. No. 6,046,056 and U.S. Pat. No. 6,576,478).

Data generated by detection of biomarkers can be analyzed with the use of a programmable digital computer. The computer program analyzes the data to indicate the number of markers detected and the strength of the signal. Data analysis can include steps of determining signal strength of a biomarker and removing data deviating from a predetermined statistical distribution. For example, the data can be normalized relative to some reference. The computer can transform the resulting data into various formats for display, if desired, or further analysis.

### Artificial Neural Network

In some embodiments, a neural network is used. A neural network can be constructed for a selected set of markers. A neural network is a two-stage regression or classification model. A neural network has a layered structure that includes a layer of input units (and the bias) connected by a layer of weights to a layer of output units. For regression, the layer of output units typically includes just one output unit. However, neural networks can handle multiple quantitative responses in a seamless fashion.

In multilayer neural networks, there are input units (input layer), hidden units (hidden layer), and output units (output layer). There is, furthermore, a single bias unit that is connected to each unit other than the input units. Neural networks are described in Duda et al., 2001, Pattern Classification, Second Edition, John Wiley &amp; Sons, Inc., New York; and Hastie et al., 2001, The Elements of Statistical Learning, Springer-Verlag, New York.

The basic approach to the use of neural networks is to start with an untrained network, present a training pattern, e.g., marker profiles from patients in the training data set, to the input layer, and to pass signals through the net and determine the output, e.g., the prognosis of the patients in the training data set, at the output layer. These outputs are then compared to the target values, e.g., actual outcomes of the patients in the training data set; and a difference corresponds to an error. This error or criterion function is some scalar function of the weights and is minimized when the network outputs match the desired outputs. Thus, the weights are adjusted to reduce this measure of error. For regression, this error can be sum-of-squared errors. For classification, this error can be either squared error or cross-entropy (deviation). See, e.g., Hastie et al., 2001, The Elements of Statistical Learning, Springer-Verlag, New York.

Three commonly used training protocols are stochastic, batch, and on-line. In stochastic training, patterns are chosen randomly from the training set and the network weights are updated for each pattern presentation. Multilayer nonlinear networks trained by gradient descent methods such as stochastic back-propagation perform a maximum-likelihood estimation of the weight values in the model defined by the network topology. In batch training, all patterns are presented to the network before learning takes place. Typically, in batch training, several passes are made through the training data. In online training, each pattern is presented once and only once to the net.

In some embodiments, consideration is given to starting values for weights. If the weights are near zero, then the operative part of the sigmoid commonly used in the hidden layer of a neural network (see, e.g., Hastie et al., 2001, The Elements of Statistical Learning, Springer-Verlag, New York) is roughly linear, and hence the neural network collapses into an approximately linear model. In some embodiments, starting values for weights are chosen to be random values near zero. Hence the model starts out nearly linear, and becomes nonlinear as the weights increase. Individual units localize to directions and introduce nonlinearities where needed. Use of exact zero weights leads to zero derivatives and perfect symmetry, and the algorithm never moves. Alternatively, starting with large weights often leads to poor solutions.

Since the scaling of inputs determines the effective scaling of weights in the bottom layer, it can have a large effect on the quality of the final solution. Thus, in some embodiments, at the outset all expression values are standardized to have mean zero and a standard deviation of one. This ensures all inputs are treated equally in the regularization process, and allows one to choose a meaningful range for the random starting weights. With standardization inputs, it is typical to take random uniform weights over the range sigma −0.7, +0.7 sigma

A recurrent problem in the use of networks having a hidden layer is the optimal number of hidden units to use in the network. The number of inputs and outputs of a network are determined by the problem to be solved. For the methods disclosed herein, the number of inputs for a given neural network can be the number of markers in the selected set of markers.

The number of outputs for the neural network will typically be just one: yes or no. However, in some embodiment more than one output is used so that more than two states can be defined by the network.

Software used to analyze the data can include code that applies an algorithm to the analysis of the signal to determine whether the signal represents a peak in a signal that corresponds to a biomarker according to the present invention. The software also can subject the data regarding observed biomarker signals to classification tree or ANN analysis, to determine whether a biomarker or combination of biomarker signals is present that indicates patient's disease diagnosis or status.

Thus, the process can be divided into the learning phase and the classification phase. In the learning phase, a learning algorithm is applied to a data set that includes members of the different classes that are meant to be classified, for example, data from a plurality of samples from patients diagnosed as PsA and who respond to anti-TNFα therapy and data from a plurality of samples from patients with a negative outcome, PsA patients who did not respond to anti-TNFα therapy. The methods used to analyze the data include, but are not limited to, artificial neural network, support vector machines, genetic algorithm and self-organizing maps, and classification and regression tree (CART) analysis. These methods are described, for example, in WO01/31579, May 3, 2001 (Barnhill et al.); WO02/06829, Jan. 24, 2002 (Hitt et al.) and WO02/42733, May 30, 2002 (Paulse et al.). The learning algorithm produces a classifying algorithm keyed to elements of the data, such as particular markers and specific concentrations of markers, usually in combination, that can classify an unknown sample into one of the two classes, e.g., responder on non-responder. The classifying algorithm is ultimately used for predictive testing.

Software, both freeware and proprietary software, is readily available to analyze patterns in data, and to devise additional patterns with any predetermined criteria for success.

### Kits

In another aspect, the present invention provides kits for determining which PsA patients will respond or not respond to treatment with an anti-TNFα agent, such as golimumab, which kits are used to detect serum markers according to the invention. The kits screen for the presence of serum markers and combinations of markers that are differentially present in PsA patients.

In one aspect, the kit contains a means for collecting a sample, such as a lance or piercing tool for causing a “stick” through the skin. The kit may, optionally, also contain a probe, such as a capillary tube, or blood collection tube for collecting blood from the stick.

In one embodiment, the kit comprises a substrate having one or more biospecific capture reagents for binding a marker according to the invention. The kit may include more than type of biospecific capture reagents, each present on the same or a different substrate.

In a further embodiment, such a kit can comprise instructions for suitable operational parameters in the form of a label or separate insert. For example, the instructions may inform a consumer how to collect the sample or how to empty or wash the probe. In yet another embodiment the kit can comprise one or more containers with biomarker samples, to be used as standard(s) for calibration.

In the method of using the algorithm of the invention for predicting the response of a PsA patient to anti-TNF therapy, blood or other fluid is acquired from the patient prior to anti-TNF therapy and at specified periods after therapy is initiated. The blood may be processed to extract a serum or plasma fraction or may be used whole. The blood or serum samples may be diluted, for example 1:2, 1:5, 1:10, 1:20, 1:50, or 1:100, or used undiluted. In one format, the serum or blood sample is applied to a prefabricated test strip or stick and incubated at room temperature for a specified period of time, such as 1 min, 5 min, 10 min, 15, min, 1 hour, or longer. After the specified period of time for the assay; the samples and the result are readable directly from the strip. For example, the results appear as varying shades of colored or gray bands, indicating a concentration range of one or more markers. The test strip kit will provide instructions for interpreting the results based on the relative concentrations of the one or more markers. Alternatively, a device capable of detecting the color saturation of the marker detection system on the strip can be provided, which device may optionally provide the results of the test interpretation based on the appropriate diagnostic algorithm for that series of markers.

### Methods of Using the Invention

The invention provides a method of predicting responsiveness to therapy with an anti-TNFα agent, such as golimumab, by analyzing detected biomarkers in a patient diagnosed with PsA. In the method of the invention, a patient is first diagnosed with PsA by an experienced professional using subjective and objective criteria.

Psoriatic arthritis is a chronic, inflammatory, usually rheumatoid factor (RF)-negative arthritis that is associated with psoriasis. The prevalence of psoriasis in the general Caucasian population is approximately 2% (Boumpas et al., 2001). Approximately 6% to 39% of psoriasis patients develop PsA (Shbeeb et al., 2000; Leonard et al., 1978). Affecting men and women equally, PsA peaks between the ages of 30 and 55 years (Boumpas, et al., 2001). Psoriatic arthritis involves peripheral joints, axial skeleton, sacroiliac joints, nails, and entheses, and is associated with psoriatic skin lesions (Gladman et al., 1987, Boumpas, et al., 2001). The presentation of PsA can be categorized into 5 overlapping clinical patterns, which include oligoarthritis in approximately 22% to 37% of patients; polyarthritis in 36% to 41% of patients; arthritis of distal interphalangeal (DIP) joints in up to 20% of patients; spondylitis affecting approximately 7% to 23% of patients; and arthritis mutilans in approximately 4% of patients (Gladman et al., 1987; Torre Alonso et al., 1991). Over one-third of patients with PsA also develop dactylitis and enthesitis (Gladman et al., 1987; Sokoll and Helliwell, 2001). Dactylitis is a painful swelling of the whole digit caused by inflammation of the digital joints and tenosynovitis.

Enthesitis is an inflammation of the tendon, ligament or joint capsule insertion into the bone. More than one-half of the patients with PsA may have evidence of erosions on x-rays, and up to 40% of the patients develop severe, erosive arthropathy (Torre Alonso et al., 1991; Gladman et al., 1987). Psoriatic arthritis leads to functional impairment, reduced quality of life, and increased mortality (Torre Alonso et al., 1991; Sokoll and Helliwell, 2001; Wong et al., 1997; Gladman et al., 1998).

Most of the treatments currently used for PsA were adapted from experience in the rheumatoid arthritis (RA) patient population. Despite the progressive and potentially disabling nature of PsA, and in contrast with RA, only a few, randomized, controlled trials have examined the role of traditional disease modifying antirheumatic drugs (DMARDs) in the treatment of PsA (Dougados et al., 1995; Jones et al., 1997; Salvarani et al., 2001; Kaltwasser et al., 2004). In these studies, methotrexate (MTX), cyclosporine, sulfasalazine, and leflunomide demonstrated efficacy in the treatment of this condition, although the treatments were associated with a time lag of several weeks between treatment initiation and a clinically significant response in either arthritis or psoriasis (MTX, cyclosporine), or only had modest efficacy on the skin (sulfasalazine, leflunomide). Corticosteroids are rarely used to treat PsA as severe psoriasis flares occur upon withdrawal.

### Clinical Assessment Methods

Psoriatic arthritis is a rheumatic condition (a disease of the joints) and is often seen in combination with skin that is red, dry, and scaly (psoriatic skin lesions). Psoriatic arthritis is a systemic rheumatic disease that can also cause inflammation in body tissues away from the joints other than the skin, such as in the eyes, heart, lungs, and kidneys. Psoriatic arthritis shares many features with several other arthritic conditions, such as ankylosing spondylitis, reactive arthritis (formerly Reiter's syndrome), and arthritis associated with Crohn's disease and ulcerative colitis. All of these conditions can cause inflammation in the spine and other joints, and the eyes, skin, mouth, and various organs. In view of their similarities and tendency to cause inflammation of the spine, these conditions are collectively referred to as “spondyloarthropathies.”

The diagnosis of PsA is most often made by assessing swollen and painful joints and certain serum markers as detailed below.

Once the diagnosis of PsA is established, the physician generally monitors clinical outcomes longitudinally in order to identify patients at risk of worsening disease.

ACR responses are presented as the numerical improvement in multiple disease assessment criteria. For example, an ACR 20 response (Felson et al., Arthr Rheum 38(6):727-735, 1995) is defined as 20% improvement in:

1. Swollen joint count (66 joints) and tender joint count (68 joints); and

2. a ≧20% improvement in 3 of the following 5 assessments
a. Patient's assessment of pain (VAS)
b. Patient's global assessment of disease activity (VAS)
c. Physician's global assessment of disease activity (VAS)
d. Patient's assessment of physical function as measured by the HAQ
e. CRP

ACR 50 and ACR 70 are similarly defined, but with a ≧50% or ≧70% improvements, respectively in these criteria.

The ACR-N Index of Improvement (Schiff et al., 1999 Arthritis Rheum. 42(Suppl 9):S81; Bathon et al., 2000 N Engl J Med. 343(22):1586-1593; Siegel and Zhen, 2005 Arthritis Rheum 52(6):1637-1641) is defined as the minimum of the following 3 items:

1. The percent improvement from baseline in tender joint counts

2. The percent improvement from baseline in swollen joint counts

3. The median percent improvement from baseline for the following 5 assessments:
a. Patient's assessment of pain (VAS)
b. Patient's global assessment of disease activity (VAS)
c. Physician's global assessment of disease activity (VAS)
d. Patient's assessment of physical function as measured by the HAQ
e. CRP

The Disease Activity Index Score 28 (DAS28) is a statistically derived index combining tender joints (28 joints), swollen joints (28 joints), CRP, and Global Health (GH) (van der Linden, 2004 available on the internet). The DAS28 is a continuous parameter and is defined as follows:

DAS28=0.56*SQRT(TEN28)+0.28*SQRT(SW28)+0.36*Ln(CRP+1)+0.014*GH+0.96

TEN28 is 28 joint count for tenderness.

SW28 is 28 joint count for swelling. The set of 28 joint count is based on left and right shoulder, elbow, wrist, metacarpo-phalangeal (MCP)1, MCP2, MCP3, MCP4, MCP5, proximal interphalangeal (PIP)1, PIP2, PIP3, PIP4, PIP5 joints of upper extremities and left and right knee joints of lower extremities.

Ln (CRP+1) is natural logarithm of (CRP value+1)

GH is Patient's Global Assessment of Disease Activity evaluated using VAS of 100 mm.

To be classified as DAS28 responder, subjects should have a good or moderate response. The DAS28 response criteria are defined in Table 1 below (van Riel, van Gestel, and Scott, 2000 EULAR Handbook of Clinical Assessments in Rheumatoid Arthritis. Alphen Aan Den Rijn, The Netherlands: Van Zuiden Communications B.V.; Ch. 40).

TABLE 1

| Present DAS28 | Improvement in DAS28 score | | |
|---|---|---|---|
| score | >1.2 | >0.6 to ≦1.2 | ≦0.6 |
| ≦3.2 | Good response | Moderate response | No response |
| >3.2 to ≦5.1 | Moderate response | Moderate response | No response |
| >5.1 | Moderate response | No response | No response |

Subjects are considered to achieve Psoriatic Arthritis Response Criteria (PsARC) if they have improvement in at least 2 (1 of which must be tender or swollen joint score) and worsening in none of the following assessments (Clegg et al., 1996 Arthritis Rheum. 39(12):2013-2020):
Patient global assessment of the disease on a 1 to 5 Likert scale (improvement=decrease by ≧1 category; worsening=increase by ≧1 category).
Physician global assessment of the disease on a 1 to 5 Likert scale (improvement=decrease by ≧1 category; worsening=increase by ≧1 category).
Tender joint score (improvement=decrease by ≧30%; worsening=increase by ≧30%).
Swollen joint score (improvement=decrease by ≧30%; worsening=increase by ≧30%).

The modified van der Heijde-Sharp score is the original vdH-S score (van der Heijde et al., 1992 Arthritis Rheum 35(1):26-34) modified for the purpose of PsA radiological damage assessment by also assessing the DIP joints of the hands. The joint erosion score is a summary of erosion severity in 40 joints of the hands and 12 joints in the feet. Each hand joint is scored, according to surface area involved, from 0 indicating no erosion and 5 indicating extensive loss of bone from more than one half of the articulating bone. Because each side of the foot joint is graded on this scale, the maximum erosion score for a foot joint is 10. Thus, the maximal erosion score is 320. The joint space narrowing (JSN) score summarizes the severity of JSN in 40 joints in the hands and 12 joints of the feet. Assessment of JSN is scored from 0 to 4, with 0 indicating no JSN and with 4 indicating complete loss of joint space, bony ankylosis, or complete luxation. Thus, the maximal JSN score is 208, and 528 is the worst possible modified vdH-S score.

The PASI is a system used for assessing and grading the severity of psoriatic lesions and their response to therapy (Fredriksson and Pettersson, 1978 Dermatologica 157(4):238-244). The PASI produces a numeric score that can range from 0 to 72. The severity of disease is calculated using a system where the body is divided in to four regions: the head (h), trunk (t), upper extremities (u), and lower extremities (1), which account for 10%, 30%, 20%, and 40% of total body surface area (BSA), respectively. Each of these areas is assessed separately for erythema, induration, and scaling, which are each rated on a scale of 0 to 4.

The scoring system of the signs of the disease (erythema, induration, and scaling) are: 0=none, 1=slight, 2=moderate, 3=severe, and 4=very severe.

The scale for estimating the area of involvement of psoriatic lesions is 0=no involvement, 1=1% to 9% involvement, 2=10% to 29% involvement, 3=30% to 49% involvement, 4=50% to 69% involvement, 5=70% to 89% involvement, and 6=90% to 100% involvement.

The PASI formula is:

PASI=0.1(Eh+Ih+Sh)Ah+0.3(Et+It+St)At+0.2(Eu+Iu+Su)Au+0.4(El+Il+Sl)Al, where E=erythema, I=induration, S=scaling, and A=area.

A prospectively identified target psoriatic lesion is evaluated for plaque induration, scaling, and erythema using the following scoring system: were erythema, 0=none, 1=light red, 2=red, but not deep red, 3=very red, 4=extremely red. Plaque induration 0=none, 1=mild (0.25 mm), 2=moderate (0.5 mm), 3=severe (1 mm), 4=very severe (1.25 mm) Scaling 0=none; 1=mainly fine scale, some of lesion covered; 2=coarser thin scale, most of lesion covered; 3=coarse thick scale, most of lesion covered, rough; 4=very thick scale, all of lesion covered, very rough.

Nail Psoriasis Severity Index (NAPSI) is based on a target fingernail representing the worst nail psoriasis, divided into quadrants and graded for nail matrix psoriasis and nail bed psoriasis (Rich and Scher, 2003 J Am Acad Dermatol. 49(2):206-212). The sum of these 2 scores is the total NAPSI score (0-8).

Nail matrix psoriasis is the presence or absence of any of the following: pitting, leukonychia, red spots in the lunula, and nail plate crumbling. Scoring for nail matrix psoriasis: 0=none, 1=present in 1/4 nail, 2=present in 2/4 nail; 3=present in 3/4 nail, 4=present in 4/4 nail.

Nail bed psoriasis is the presence or absence of any of the following: onycholysis, splinter hemorrhages, oil drop discoloration, and nail bed hyperkeratosis. The score for nail bed psoriasis is the same as for nail matrix psoriasis.

Patients may be scored using a generalized health related quality of life survey form such as the short form 36 (SF-36) (Ware J E, Jr., Snow K S, Kosinski M, Gandek B. The SF-36 health survey manual and interpretation guide. Boston: The Health Institute, New England Medical Center, 1993) which includes physical functions as well as mental aspects and can be subcategorized into a physical components score (PCS) and a mental components score (MCS).

It will be recognized that the clinical indices described herein are part of the patient data set and can be assigned a numerical score.

### Suitability for TNFα Therapy

Anti-TNFα agents have been commercially available, such as golimumab and infliximab, and used to treat PsA for several years. The efficacy and safety profile of anti-TNF therapy for a variety of indications, including PsA, has been well characterized.

### Patient Management

In the method of the invention for predicting or assessing early responsiveness to anti-TNF therapy, prior to initiation of anti-TNF therapy, at a “baseline visit”, a baseline or “Week 0” sample is acquired from the patient to be treated with anti-TNF therapy. The sample may be any tissue which can be evaluated for the biomarkers associated with the method of the invention. In one embodiment the sample is a fluid selected from the group consisting of a fluid selected from the group consisting of blood, serum, plasma, urine, semen and stool. In a particular embodiment, the sample is a serum sample which is obtained from patient's blood drawn by a standard method of direct venipuncture or via an intravenous catheter.

In addition, at the baseline visit, information on patient's demographics and history of disease with PsA will be recorded on a standardized form or case report form. Data such as time since patient's diagnosis, previous treatment history, concomitant medications, C-reactive protein (CRP) level and an assessment of disease activity (i.e., ACR or DAS28) will be recorded.

The patient receives the first dose of anti-TNF therapy at the time of the baseline visit or within 24-48 hours. At the time of the baseline visit, the patient is scheduled for a Week 4 visit.

At the 2-week visit or 4-week visit, approximately 14 or 28 days after initial administration of anti-TNFα therapy, a second patient sample is acquired, preferably using the same protocol and route as for the baseline sample. The patient is examined and other indices, imaging, or information may be performed or monitored as proscribed by the health care professional or study design as indicated. The patient is scheduled for subsequent visits, such as a Week 8, Week 12, Week 14, Week 28, etc. visit for the purposes of performing assessment of disease using the such criteria as set forth by the ACR and PsARC and for the acquisition of patient samples for biomarker evaluation.

At any or the above times prior to, during, or following treatment, other parameters and markers may be assessed in the patient's sample or other fluid or tissue samples acquired from the patient. These may include standard hematological parameters such as hemoglobin content, hematocrit, red cell volume, mean red cell diameter, erythrocyte sedimentation rate (ESR), and the like. Other markers may which have been determined useful in assessing the presence of PsA may be quantitated in some or all of the patient's sample(s), such as, CRP (Spoorenberg A et al., 1999. J Rheumatol 26: 980-984) and IL-6, and markers of cartilage degradation such as serum Type 1 N-telopeptides (NTX), urinary type II collagen C-telopeptides (urinary CTX-II) and serum matrix metalloptrotease 3 (MMP3, stromelysin 1)(See US20070172897).

The medical professional's clinical judgment of response should not be negated by the test result. However, the test could aid in making the decision to continue or discontinue treatment with golimumab. In a test in which the prediction model (algorithm) has 90% sensitivity and 60% specificity, where 50% of the patients display a clinical response and 50% do not display assessment scores or evaluations consistent with a clinical response. This would mean: of the responders, 45% would be identified correctly as responders (5 would be reported as likely non-responders) and 30% or non-responders would be identified correctly as non-responders (20% would be classified as likely responders). Thus, overall benefit is that 60% of all true non-responders could be spared an unnecessary therapy or discontinued from therapy at an early time point (Week 4). The 5% false-negative “responders” (identified as likely non-responders) would have been treated, and as with all patients, their response would be judged clinically before making the decision to continue or discontinue treatment at Week 14 or later. The 20% false-negative “non-responders” (identified as possible responders) would have to be judged clinically, and would take the usual time to make the decision to discontinue treatment.

### Example 1

### Sample Collection and Analysis

Serum samples were obtained and evaluated from patients enrolled in a multicenter, randomized, double-blind, placebo-controlled, 3-arm study (with early escape at Week 16) of placebo, golimumab 50 mg, or golimumab 100 mg administered as SC injections every 4 weeks in subjects with active PsA. Subjects were to be assessed for routine efficacy and safety assessments through Week 52, with long term follow-up through 5 years of treatment. Primary efficacy assessments were made at week 14 and week 24. The study was conducted at 57 global investigational sites and enrolled 405 subjects. Subjects may also be receiving methotrexate (MTX), NSAIDS, or oral or low potency (2.5% or less) topical corticosteroids. If receiving MTX, treatment should have started at least 3 months prior to receiving golimumab, not exceed 25 mg/week, be stable and not exhibit serious side effects attributable to MTX. Other treatments are discontinued prior to entry into the study.

At selected study sites, 100 subjects had serum samples collected for biomarker profiling and certain single analyte ELISAs. The biomarker sampling occurred at baseline and at weeks 4 and 14 on study. One of the objectives of the serum biomarker component of the study was to identify whether a biomarker (or set of biomarkers) could be used to prospectively predict a subject's response or non-response to golimumab.

Biomarker data was collected at three timepoints for each subject in the substudy: baseline, week 4, and week 14. At each time point, 92 protein biomarkers were assayed. A complete list of the biomarkers is shown in Table 2.

The sera were analyzed for biomarkers using commercially available assays employing either a multiplex analysis performed by Rules Based Medicine (Austin, Tex.) or single analyte ELISA. All samples were stored at −80° C. until tested. The samples were thawed at room temperature, vortexed, spun at 13,000×g for 5 minutes for clarification and 150 uL was removed for antigen analysis into a master microtiter plate. Using automated pipetting, an aliquot of each sample was introduced into one of the capture microsphere multiplexes of the analytes. These mixtures of sample and capture microspheres were thoroughly mixed and incubated at room temperature for 1 hour. Multiplexed cocktails of biotinylated, reporter antibodies for each multiplex were used and detected using streptavidin-phycoerythrin. Analysis was performed in a Luminex 100 instrument and the resulting data stream was interpreted using proprietary data analysis software developed at Rules-Based Medicine and licensed to Qiagen Instruments. For each multiplex, both calibrators and controls were run. Testing results were determined first for the high, medium and low controls for each multiplex to ensure proper assay performance. Unknown values for each of the analytes localized in a specific multiplex were determined using 4 and 5 parameter, weighted and non-weighted curve fitting algorithms included in the data analysis package.

TABLE 2

| | | Swiss-Prot |
|---|---|---|
| Protein Biomarker | Units | Accession # |
| Adiponectin | ug/mL | Q15848 |
| Alpha-1 Antitypsin | mg/mL | P07758 |
| Alpha-2 Macroglobulin | mg/mL | P01023 |
| Alpha-Fetoprotein | ng/mL | P02771 |
| Apolipoprotein A-1 | mg/mL | P02647 |
| Apolipoprotein CIII | ug/mL | P02656 |
| Apolipoprotein H | ug/mL | P02749 |
| Beta 2-Microglobulin | ug/mL | P01884 |
| Brain-Derived Neurotrophic Factor | ng/mL | P23560 |
| (BDNF) | | |
| Calcitonin | pg/mL | P01258 |
| Cancer Antigen 125 | U/mL | Q14596 |
| Cancer Antigen 19-9 | U/mL | Q9BXJ9 |
| Carcinoembryonic Antigen | ng/mL | P78448 |
| CD40 | ng/mL | P25942 |
| CD40 Ligand | ng/mL | P29965 |
| Complement component 3 | mg/mL | P01024 |
| C-Reactive Protein | ug/mL | P02741 |
| Creatine Kinase MB - Brain | ng/mL | P12277 |
| ENA-78 | ng/mL | P42830 |
| (Epithelial Neutrophil Activating | | |
| Peptide 78) | | |
| Endothelin | pg/mL | P05305 |
| ENRAGE | ng/mL | P80511 |
| Eotaxin | pg/mL | P51671 |
| Epidermal Growth Factor | pg/mL | P01133 |
| Erythropoietin | pg/mL | P01588 |
| Factor VII | ng/mL | P08709 |
| Fatty Acid Binding Protein | ng/mL | P05413 |
| Ferritin - Heavy | ng/mL | P02794 |
| FGF-basic | pg/mL | P09038 |
| Fibrinogen alpha chain | mg/mL | P02671 |
| G-CSF | pg/mL | P09919 |
| Glutathione S-Transferase alpha | ng/mL | P08263 |
| GM-CSF | pg/mL | P04141 |
| Growth Hormone | ng/mL | P01241 |
| Haptoglobin | mg/mL | P00738 |
| ICAM-1 (Intercellular Adhesion | ng/mL | P05362 |
| Molecule 1) | | |
| IFN gamma | pg/mL | P01579 |
| IgA | mg/mL | na |
| IgE | ng/mL | na |
| IGF-1 | ng/mL | P05019 |
| IgM | mg/mL | na |
| IL-1 receptor antagonist | pg/mL | Q9UBH0 |
| IL-10 | pg/mL | P22301 |
| IL-12 p40 | ng/mL | P29460 |
| IL-12 p70 | pg/mL | P29459 |
| IL-13 | pg/mL | P35225 |
| IL-15 | ng/mL | P40933 |
| IL-16 | pg/mL | Q14005 |
| IL-17 (IL17A) | pg/mL | Q16552 |
| IL-18 | pg/mL | Q14116 |
| IL-1alpha | ng/mL | P01583 |
| IL-1beta | pg/mL | P01584 |
| IL-2 | pg/mL | P01585 |
| IL-23 p19 | ng/mL | Q9NPF7 |
| IL-3 | ng/mL | P08700 |
| IL-4 | pg/mL | P05112 |
| IL-5 | pg/mL | P05113 |
| IL-6 | pg/mL | P05231 |
| IL-7 | pg/mL | P13232 |
| IL-8 | pg/mL | P10145 |
| Insulin | uIU/mL | P01308 |
| Leptin | ng/mL | P41159 |
| Lipoprotein (a) | ug/mL | P08519 |
| Lymphotactin | ng/mL | P47992 |
| MCP-1 (Monocyte Chemotactic Protein | pg/mL | P13500 |
| 1) | | |
| MDC (Macrophage-Derived Chemokine) | pg/mL | O00626 |
| MIP-1 alpha (Macrophage Inflammatory | pg/mL | P10147 |
| Protein 1 alpha) | | |
| MIP-1 beta (Macrophage Inflammatory | pg/mL | P13236 |
| Protein 1 beta) | | |
| MMP-2 (Matrix Metalloproteinase 2) | ng/mL | P08253 |
| MMP-3 (Matrix Metalloproteinase 3) | ng/mL | P08254 |
| MMP-9 (Matrix Metalloproteinase 9) | ng/mL | P14780 |
| Myeloperoxidase | ng/mL | P05164 |
| Myoglobin | ng/mL | P02144 |
| PAI-1 | ng/mL | P05121 |
| PAPPA | mIU/mL | Q13219 |
| Prostate-Specific Antigen (PSA), Free | ng/mL | P07288 |
| Prostatic Acid Phosphatase (PAP) | ng/mL | P15309 |
| RANTES | ng/mL | P13501 |
| serum amyloid P component, (SA) | ug/mL | P02743 |
| SGOT (Serum Glutamic Oxaloacetic | ug/mL | P17174 |
| Transaminase) | | |
| SHBG | nmol/L | P04278 |
| Stem Cell Factor | pg/mL | P21583 |
| Thrombopoietin (TPO) | ng/mL | P40225 |
| Thyroid Stimulating Hormone (TSH) - | uIU/mL | P01215 |
| alpha | | |
| Thyroxine Binding Globulin (TBG) | ug/mL | P05543 |
| TIMP-1 (Tissue Inhibitor of | ng/mL | P01033 |
| Metalloproteinase 1) | | |
| Tissue factor (coagulation factor III, | ng/mL | P13726 |
| thromboplastin) | | |
| TNF RII (Tumor Necrosis Factor | ng/mL | Q92956 |
| Receptor 2) | | |
| TNF-alpha (Tumor Necrosis Factor | pg/mL | P01375 |
| alpha) | | |
| TNF-beta (Tumor Necrosis Factor beta) | pg/mL | P01374 |
| VCAM-1 | ng/mL | P19320 |
| VEGF | pg/mL | P15692 |
| vWF (von Willebrand Factor) | ug/mL | P04275 |

All 100 subjects enrolled in the sub-study had complete protein biomarker data collected for all three timepoints (baseline, week 4, and week 14), for a total of 300 subject samples.

Each of the 92 biomarkers has an established lower limit of quantification (LLOQ). The Biomarker statistical analysis plan (SAP) prospectively defined a criterion for using a biomarker in the analysis that required the biomarker to be above the limit of quantification in at least 20% of baseline samples. Of the 92 biomarkers, 62 (67%) met that criterion for inclusion in the subsequent analysis. The distribution of the number of samples at the lower limit of detection across biomarkers was plotted. Table 3 identifies the biomarkers that were included in the final analysis. An assessment of the distributions of each biomarker was made to determine whether a log transformation of that biomarker was warranted. This assessment was made without regard to treatment group. Overall, 59 of the 62 biomarkers in the analysis set were log 2 transformed (Table 3).

TABLE 3

| | | | #Samples | Log |
|---|---|---|---|---|
| | | | at LOQ | Trans- |
| Marker | Units | LOQ | (300 Total) | form |
| Adiponectin | ug/mL | 0.2 | 0 | TRUE |
| Alpha-1 Antitrypsin | mg/mL | 0.011 | 0 | TRUE |
| Alpha-2 Macroglobulin | mg/mL | 0.061 | 0 | TRUE |
| Alpha-Fetoprotein | ng/mL | 0.43 | 6 | TRUE |
| Apolipoprotein A1 | mg/mL | 0.0066 | 0 | TRUE |
| Apolipoprotein CIII | ug/mL | 2.7 | 0 | TRUE |
| Apolipoprotein H | ug/mL | 8.8 | 0 | TRUE |
| Beta-2 Microglobulin | ug/mL | 0.013 | 0 | TRUE |
| Brain-Derived Neurotrophic | ng/mL | 0.029 | 0 | TRUE |
| Factor | | | | |
| C Reactive Protein | ug/mL | 0.0015 | 0 | TRUE |
| Cancer Antigen 125 | U/mL | 4.2 | 0 | TRUE |
| Cancer Antigen 19-9 | U/mL | 0.25 | 27 | TRUE |
| Carcinoembryonic Antigen | ng/mL | 0.84 | 127 | TRUE |
| CD40 | ng/mL | 0.021 | 0 | TRUE |
| CD40 Ligand | ng/mL | 0.02 | 0 | FALSE |
| Complement 3 | mg/mL | 0.0053 | 0 | TRUE |
| EGF | pg/mL | 7.4 | 13 | TRUE |
| EN-RAGE | ng/mL | 0.25 | 0 | TRUE |
| ENA-78 | ng/mL | 0.076 | 0 | TRUE |
| Eotaxin | pg/mL | 41 | 17 | TRUE |
| Factor VII | ng/mL | 1 | 0 | TRUE |
| Ferritin | ng/mL | 1.4 | 0 | TRUE |
| Fibrinogen | mg/mL | 0.0098 | 120 | TRUE |
| G-CSF | pg/mL | 5 | 117 | TRUE |
| Glutathione S-Transferase | ng/mL | 0.4 | 0 | TRUE |
| Growth Hormone | ng/mL | 0.13 | 159 | TRUE |
| Haptoglobin | mg/mL | 0.025 | 1 | TRUE |
| ICAM-1 | ng/mL | 3.2 | 0 | TRUE |
| IgA | mg/mL | 0.0084 | 5 | FALSE |
| IgE | ng/mL | 14 | 213 | TRUE |
| IGF-1 | ng/mL | 4 | 180 | TRUE |
| IgM | mg/mL | 0.015 | 0 | TRUE |
| IL-16 | pg/mL | 66 | 0 | TRUE |
| IL-18 | pg/mL | 54 | 1 | TRUE |
| IL-1ra | pg/mL | 15 | 10 | TRUE |
| IL-8 | pg/mL | 3.5 | 3 | TRUE |
| Insulin | uIU/mL | 0.86 | 24 | TRUE |
| Leptin | ng/mL | 0.1 | 0 | TRUE |
| Lipoprotein (a) | ug/mL | 3.7 | 0 | TRUE |
| MCP-1 | pg/mL | 52 | 2 | TRUE |
| MDC | pg/mL | 14 | 0 | TRUE |
| MIP-1alpha | pg/mL | 13 | 183 | TRUE |
| MIP-1beta | pg/mL | 38 | 4 | TRUE |
| MMP-3 | ng/mL | 0.2 | 0 | TRUE |
| Myeloperoxidase | ng/mL | 68 | 14 | TRUE |
| Myoglobin | ng/mL | 1.1 | 0 | TRUE |
| PAI-1 | ng/mL | 0.9 | 0 | TRUE |
| Prostate Specific Antigen, | ng/mL | 0.023 | 117 | TRUE |
| Free | | | | |
| Prostatic Acid Phosphatase | ng/mL | 0.034 | 0 | TRUE |
| RANTES | ng/mL | 0.048 | 0 | TRUE |
| Serum Amyloid P (SAP) | ug/mL | 0.058 | 0 | TRUE |
| SGOT | ug/mL | 3.7 | 58 | TRUE |
| SHBG | nmol/L | 1.3 | 0 | TRUE |
| Stem Cell Factor | pg/mL | 56 | 0 | TRUE |
| Thyroid Stimulating Hormone | uIU/mL | 0.028 | 3 | FALSE |
| Thyroxine Binding Globulin | ug/mL | 0.34 | 0 | TRUE |
| TIMP-1 | ng/mL | 8.4 | 0 | TRUE |
| TNF-alpha | pg/mL | 4 | 242 | TRUE |
| TNF RII | ng/mL | 0.13 | 0 | TRUE |
| VCAM-1 | ng/mL | 2.6 | 0 | TRUE |
| VEGF | pg/mL | 7.5 | 0 | TRUE |
| von Willebrand Factor | ug/mL | 0.4 | 0 | TRUE |

A clustered correlation (heatmap) was used as an overall assessment of data quality. No sample outliers were seen in that analysis. The average pairwise correlation from the sample correlation matrix was also assessed and all samples showed at least an average of 89% correlation to other samples, indicating the biomarker data was consistent across subject samples.

Thus, the quality of the data was assessed as very high for the biomarker protein profiling analysis. No samples were excluded and 62 of the 92 biomarkers measured had detectable (20% of samples above the LLOQ) data available for inclusion in the analysis.

### Example 2

### Clinical Endpoint and Data Validation

The data from 100 patients representing a subgroup of a 405 patient clinical study of golimumab in the treatment of psoriatic arthritis were analyzed using biometric, clinical assessment measurements and the 62 biomarker values.

Baseline clinical characteristics for subjects in the substudy were well balanced across the three treatment groups (Table 4) where continuous variables are represented as the Mean±SD (Min-Max) and categorical variables as percentages. Note that this CRP measurement was obtained separately from the CRP generated on the protein array. All subjects in the substudy were followed through weeks 14 and 24 and had each of the protocol-specified biomarker assessments at three time points (baseline, Week 4, and Week 14). While some subjects qualified for the early escape phase of the trial (had less than 10% improvement in tender and swollen joint count at week 16), all subjects had clinical endpoint data at 14 and 24 weeks (Table 5).

TABLE 4

| | Placebo | Gol 50 mg | Gol 100 mg | Total |
|---|---|---|---|---|
| N | 26 | 39 | 35 | 100 |
| Age (yrs) | 44.3 ± 10.7 | 46.9 ± 10.0 | 50.7 ± 9.8 | 47.5 ± 10.3 |
| | (29-66) | (29-68) | (29-77) | (29-77) |
| Weight (kg) | 87.2 ± 19.6 | 91.3 ± 16.6 | 92.9 ± 20.6 | 90.8 ± 18.8 |
| | (59-136) | (55-126) | (61-144) | (55-144) |
| Sex (% Male) | 54% | 67% | 60% | 61% |
| Race | 96% | 92% | 94% | 94% |
| (% Caucasian) | | | | |
| CRP¹ | 1.19 ± 1.40 | 1.03 ± 1.26 | 1.63 ± 1.94 | 1.28 ± 1.57 |
| (ug/mL) | (0.3-5.1) | (0.3-6.9) | (0.3-9.2) | (0.30-9.20) |
| MTX Usage | 38% | 33% | 37% | 36% |
| (% Yes) | | | | |
| Swollen Joint | 11.8 ± 8.7 | 13.0 ± 7.4 | 10.3 ± 4.9 | 11.7 ± 7.0 |
| Count | (3-43) | (3-43) | (3-22) | (3-43) |
| Tender Joint | 20.7 ± 12.5 | 21.1 ± 13.0 | 21.0 ± 10.5 | 21.0 ± 12.0 |
| Count | (6-55) | (3-50) | (3-52) | (3-55) |

TABLE 5

| | Enrolled | Baseline | Week 4 | Week 14 | Qualified for | Clinical Endpoint |
|---|---|---|---|---|---|---|
| Treatment | in Protein | Data | Data | Data | Early Escape at | Data Available |
| Group | Study | Collected | Collected | Collected | Week 16 | at Weeks 14/24 |
| Placebo | 26 | 26/26 (100%) | 26/26 (100%) | 26/26 (100%) | 11/26 (42%) | 26/26 (100%) |
| Gol 50 mg | 39 | 39/39 (100%) | 39/39 (100%) | 39/39 (100%) | 6/39 (15%) | 39/39 (100%) |
| Gol 100 mg | 35 | 35/35 (100%) | 35/35 (100%) | 35/35 (100%) | 7/35 (20%) | 35/35 (100%) |
| Total | 100 | 100/100 (100%) | 100/100 (100%) | 100/100 (100%) | 24/100 (24%) | 100/100 (100%) |

The treatment effect on clinical endpoints within this cohort, is shown in Table 6 (responder/total in each group). The golimumab groups had significantly higher response rates compared to placebo across the range of clinical endpoints assessed, with the exception of HAQ.

TABLE 6

| | | | | | Gol vs |
|---|---|---|---|---|---|
| Endpoint | Gol 100 mg | Gol 50 mg | Placebo | Overall | Placebo p |
| ACR20 Wk 14 | 13/35 (37%) | 21/39 (54%) | 2/26 (8%) | 36/100 (36%) | 0.0003 |
| ACR20 Wk 24 | 24/35 (69%) | 19/39 (49%) | 6/26 (23%) | 49/100 (49%) | 0.003 |
| DAS28 Wk 14 | 24/35 (69%) | 26/39 (67%) | 6/26 (23%) | 56/100 (56%) | 0.0002 |
| DAS28 Wk 24 | 29/35 (83%) | 26/39 (67%) | 7/26 (27%) | 62/100 (62%) | 0.00004 |
| PASI75 Wk 14 | 14/35 (40%) | 11/39 (28%) | 3/26 (12%) | 28/100 (28%) | 0.041 |
| ΔPCS Wk 14 | 23/35 (66%) | 22/39 (56%) | 6/26 (23%) | 51/100 (51%) | 0.001 |
| HAQ Wk 14 | 22/35 (63%) | 23/39 (59%) | 10/26 (38%) | 55/100 (55%) | 0.067 |
| HAQ Wk 24 | 23/35 (66%) | 19/39 (49%) | 11/26 (42%) | 53/100 (53%) | 0.256 |

After the initial analysis of changes in markers levels by treatment group it was clear that there was no dose response effect. Thus it was decided to combine the golimumab treatment groups.

### Example 3

### Model Building

At baseline, there were multiple significant associations between biomarker levels and biometric or clinical characteristics of sex, weight, age, baseline CRP, baseline swollen joint count (SJC.bl), and tender joint count at baseline (TJC.bl) found by robust linear regression analysis. For example, leptin correlated with sex, weight, and age with a p-value of less than 0.01.

Markers that changed between baseline and Week 4, where the change was significantly (p<0.01) different between the placebo group and golimumab treated group include: alpha-1-Antitrypsin, CRP, ENRAGE, haptoglobin, ICAM-1, IL-16, IL-18, IL-1ra, IL-8, MCP-1, MIP-1beta, MMP-3, myeloperoxidase, serum amyloid P, thyroxine binding globulin, TNFRII, and VEGF.

The clinical study demonstrated that golimumab treatment was significantly superior to placebo across the range of clinical endpoints assessed for subjects with PsA, with the exception of HAQ. Robust logistic regression models were used to test for the association of biomarkers with clinical endpoints. Predictive models were developed using a classification and regression tree (CART) approach with cross validation.

A series of statistical analyses was performed to determine if there was an association between biomarker expression and the primary clinical endpoints, within the combined golimumab treated group.

All analysis was performed using R (R: A Language and Environment for Statistical Computing, 2008, Author: R Development Core Team, R Foundation for Statistical Computing, Vienna, Austria, ISBN 3-900051-07-0). Change from baseline was tested using one-sample t-tests. Association of clinical factors with baseline biomarkers was evaluated using robust linear regression models. Robust logistic regression models were used to test for the association of biomarkers with clinical endpoints. Clinical endpoint variables that were Yes/No used a 1/0 coding. Clinical endpoints that were continuous were converted into 1/0 variables for this analysis by applying a threshold at the median value of all subjects.

Generally, the identification of markers associated with the different clinical endpoints varied across endpoints. This result is most likely due to the differences in the clinical endpoint measures, i.e., ACR measures arthritis related signs and symptoms whereas PASI measures changes in the skin. The endpoint with the strongest set of biomarker associations was DAS28, at both week 14 and week 24. DAS28 was also the endpoint with the most significant treatment effect.

Since many comparisons were made in this analysis (62 markers at baseline, wk 4, wk 14, as well as change in marker from baseline to week 4, and change from baseline to week 14 times the 9 clinical endpoints), using a p value of <0.05 for a marker association (odds ratio) with a single endpoint at a single time point was not considered to be sufficiently strong evidence for an association. To increase the reliability of the results, the focus was put on identifying markers that showed significant association with multiple clinical endpoints at multiple timepoints. The baseline markers identified consistently across timepoints and clinical endpoints were: adiponectin, prostatic acid phosphatase (PAP), MDC (also described as macrophage-derived chemokine, MDC(1-69), MGC34554, CCL22, SCYA22, small inducible cytokine A22 precursor, STCP-1, stimulated T-cell chemotactic protein 1), SGOT (aspartate aminotransferase), and VEGF. Each of these five markers was significant for at least four clinical endpoints, was significant for at least three timepoints, and had an odds ratio (OR) of greater than 2.0 for at least one endpoint. For these markers, Table 7 shows the odds ratios and p-values for biomarker association with the clinical endpoint DAS28 for all golimumab treated subjects. In this table, the OR represents the increased odds of a clinical response for a 1 unit change on the log 2 scale, or a doubling on the linear scale. Numbers less than 1 represent an inverse association.

TABLE 7

| | | | Δ | | | | Δ | | | |
|---|---|---|---|---|---|---|---|---|---|---|
| | Week 0 | | Week 4 | | Week 4 | | Week 14 | | Week 14 | |
| Marker | OR | p | OR | p | OR | p | OR | p | OR | p |
| Adiponectin | 2.26 | 0.025 | 8.99 | 0.061 | 2.82 | 0.009 | 2.39 | 0.456 | 2.56 | 0.015 |
| MDC | 0.59 | 0.274 | 0.34 | 0.165 | 0.34 | 0.041 | 0.49 | 0.339 | 0.29 | 0.036 |
| PAP | 2.99 | 0.017 | 0.15 | 0.005 | 0.80 | 0.644 | 0.25 | 0.015 | 0.89 | 0.788 |
| SGOT | 0.28 | 0.002 | 2.69 | 0.023 | 0.69 | 0.269 | 2.10 | 0.046 | 0.66 | 0.277 |
| VEGF | 2.21 | 0.014 | 0.21 | 0.014 | 1.42 | 0.160 | 0.28 | 0.053 | 1.64 | 0.072 |

Table 8 shows the statistical association of these five markers across at least two endpoints either based on Week 4 or Week 14 biomarker data where 1=ACR20Wk14; 2=ACR20Wk24; 3=Early Escape; 4=DAS28 Wk14; 5=DAS28Wk24; 6=PCSWk14; 7=PASI75Wk14; 8=HAQWk14; 9=HAQWk24. In general the Week 4 and Week 14 markers were similar, and showed significant association to multiple clinical endpoints.

TABLE 8

| Marker | Week 0 | Δ Week 4 | Week 4 | Δ Week 14 | Week 14 |
|---|---|---|---|---|---|
| Adiponectin | 3, 4, 7 | 8 | 4, 7 | | 4, 7 |
| MDC | | 3, 5, 7 | 1, 4, 9 | 1, 3, 7 | 4 |
| PAP | 1, 2, 4, 5 | 1, 4, 5 | | 1, 4, 5 | |
| SGOT | 2, 4, 5, 6 | 4, 5 | 2 | 4, 7 | 2 |
| VEGF | 4 | 4, 5, 9 | | 5, 8, 9 | |

In contrast to the biomarker/clinical endpoint associations observed within the golimumab treated group, there was no association of biomarker values to clinical endpoint responses within the placebo group. This result serves as an internal control or benchmark for the more significant biomarker results seen in the golimumab biomarker analyses.

A method using statistical analyses was developed to determine which biomarkers could be used to predict the response of the patients to treatment. All markers were eligible for inclusion in the model, not just those displaying individual (univariant) statistical significance. The rationale for this approach is that certain markers may not be strongly predictive on their own, but may add predictive strength to the model after accounting for the effects of other markers.

All prediction models herein were developed using classification and regression trees (CART) and employed cross validation. The CART models are displayed in the form of a decision tree. The end nodes of the tree are labeled with a class prediction (Yes for a predicted clinical endpoint responder, No for a predicted non-responder) and two numbers (x/y, where x is the actual number of non-responders in the study who would fall into that node and y is the actual number of responders who would fall into that node). The overall accuracy of the model is the number of x's across the ‘No’ end nodes plus the number of y's across the ‘Yes’ end nodes. Models were developed for the primary clinical endpoint, ACR20, at Week 14.

First, a clinical-only model was developed, where only clinical factors (no protein biomarkers) were used to build and validate the model. The clinical model serves as a benchmark against which the various biomarker prediction models can be evaluated. Second, a model was built based on only baseline biomarker data. A third model incorporated both baseline clinical factors and baseline biomarker data. The fourth model used biomarker data at baseline and at week4 (change from baseline). The last model used biomarker data at baseline and at week4 (change from baseline) as well as clinical factors. All markers were eligible for inclusion in the model, not just markers with univariate significance.

### Clinical Only

The accuracy of the clinical-only model was 49/74 (66%) for prediction of clinical response (ACR20 at Week14). The model is displayed in FIG. 1. The clinical model uses age as the initial predictor: subjects above 50.5 years are predicted to be non-responders; subjects below 37.5 years are predicted to be responders, and subjects with intermediate age are classified based on the secondary predictor of baseline CRP (baseline CRP above 0.55 predicted as responders, baseline CRP below 0.55 predicted as non-responders). This model sensitivity was 50%, and the model specificity was 80%.

### Baseline Biomarker Prediction Models

The statistical method was applied to determine which biomarkers at baseline could be used to predict the response of the patients to treatment using ACR20 measured at Week 14. A diagram of the model is given in FIG. 2 showing that the decision tree uses VEGF analyzed by the present protein profiling method as the initial classifier: that is, patients with VEGF less than 8.082 (log scale) are predicted to be non-responders. Subjects with VEGF levels greater than or equal to 8.082 are further classified using the baseline PAP and adiponectin levels. Patients are classified as non-responders if PAP is less than or equal to 2.287 (log scale); those with baseline PAP levels greater than 2.287 are then further classified based on the use of a secondary predictor of baseline adiponectin. The patients with an adiponectin result greater than or equal to 1.35 (log scale) are predicted to be responders, while patients with adiponectin below 1.35 predicted to be non-responders. The accuracy (percentage True Positives+True Negatives) of the model overall was 76% and for predicting responders was 53% vs predicting non-responders at 95%. The sensitivity of the model was 53% and specificity 95%. Thus, using this model, the patient's clinical outcome (ACR20) at Week 14 was accurately predicted for 76% of the patients. This is considered a weak model due to the low sensitivity.

Change from Baseline at Week 4

A prediction model using the biomarker data was developed to determine if the change in a biomarker concentration at Week 4 of treatment could predict the clinical outcome at Week 14. The model is displayed in FIG. 3. The biomarker model uses the change from baseline in MDC levels as the initial classifier: patients with MDC decreases greater than or equal to −0.1206 (log scale) fall into branch 1 of the model; patients with an MDC decrease which is less than −0.1206 fall into branch 2 of the model. The patients on branch 1 are further classified based on the change in lipoprotein A. Subjects on branch 1 with change in Lipoprotein A concentration greater than or equal to −0.2275 are classified as non-responders, and those with a change <−0.2275 are responders. For those subjects in branch 2, subjects with a decrease from baseline in beta-2 microglobulin levels greater than or equal to −0.1112 are classified as responders; those with beta-2 microglobulin change less than −0.1112 are classified as non-responders. The accuracy of the model for responders was 79%, and the accuracy for non-responders was 90% (combined accuracy was 63/74 (85%) for predicting clinical outcome (ACR20) at Week 14. Sensitivity was 73% and specificity was 90%.

When the CART analysis method was performed using the baseline or change from baseline to week 4 biomarker data plus the clinical factors (sex, weight, age, baseline CRP, SJC.bl, and TJC.bl) the sensitivity and specificity of the model produced was identical to the baseline and week4 biomarker model, indicating that the clinical factors at baseline did not enhance the predictive power of the algorithm over that relying on serum markers only.

### SUMMARY

The results of the protein biomarker study showed that multiple biomarkers changed significantly as a consequence of golimumab therapy. In contrast, few biomarker changes were observed in the placebo control arm. Two novel biomarker-based clinical response prediction models were developed, one that used baseline biomarker values to predict a patients clinical response, another that used early (Week 4) changes in biomarker values to predict longer term (for example, Week 14) clinical responses. The models suggest that a subset of the markers have changes associated with clinical response to golimumab, as opposed to simply being non-specific effects of treatment, which provide a sensitive and specific predictive model (Table 9). Importantly, the biomarker values (either at baseline or the week 4 changes) preceded the longer term clinical outcomes.

TABLE 9

| Model | Accuracy | Sensitivity | Specificity |
|---|---|---|---|
| Clinical Only | 66% | 50% | 80% |
| Baseline | 76% | 53% | 95% |
| Week 4 change | 85% | 73% | 90% |
| from Baseline | | | |

Adiponectin is important for homeostasis of glucose metabolism and levels are elevated in RA patients with active disease (Popa et al., 2009). VEGF is an endothelial growth factor and plays a role in angiogenesis, a hallmark of the inflamed skin and joints of patients with active PsA (Fink et al., 2007). MDC or CCL22 is a chemokine that is elevated in patients with juvenile inflammatory arthritis (Jager et al., 2007). Elevated levels of liver enzymes (including SGOT) have been shown in rheumatoid arthritis and psoriatic arthritis patients (Curtis et al., 2009). Thus, the markers identified in the predictive algorithm may be representative of disease associated processes.

What is claimed:

1. A method for predicting the response of a psoriatic arthritis patient to anti-TNFα therapy, said method comprising:
determining the concentration of at least one serum marker selected from the group consisting of adiponectin, prostatic acid phosphatase (PAP), MDC, SGOT, VEGF, lipoprotein A and beta-2-microgloblulin; and
comparing said concentration with a cutoff value determined by analyzing a set of values of serum concentrations of the marker from patients diagnosed with psoriatic arthritis who received anti-TNFα therapy and were classified as a responder or a non-responder based on one or more clinical endpoints.

2. The method of claim 1, wherein the concentration of at least two serum markers is determined and compared with concentrations of individual cutoff values for said markers.

3. A method for predicting the response of a psoriatic arthritis patient to anti-TNFα therapy comprising:
a) obtaining a sample from the patient prior to the administration of an anti-TNFα agent at a specified time point after the initiation of anti-TNFα therapy;
b) determining the concentration of MDC, lipoprotein A and beta-20-microglobulin in the sample for each time point; and
c) comparing the change in concentration of MDC in the sample to a MDC cutoff value whereby if the concentration is determined to be greater than, or equal to said MDC cutoff value, the patient is further classified based on the change in liproprotein A values in the sample, and if the change is below the lipoprotein A cutoff value the patient is further classified based on the change in beta-2-microglobulin level in the serum between the pre-treatment sample and the post-treatment sample; whereby the values can be used to predict whether the patient will be a non-responder to anti-TNFα using clinical assessment measurements.

4. The method of claim 3, wherein the sample is serum.

5. The method of claim 4 where the change in serum MDC is log transformed and the cutoff value is −0.12.

6. The method of claim 3, wherein concentration of lipoprotein A in serum is log transformed and the change in lipoprotein A cutoff value is −0.23.

7. The method of claim 3, wherein concentration of beta-2-microglobulin in serum is log transformed and the change in beta-2-microglobulin cutoff value is −0.11.

8. The method of claim 3, wherein the determining step is performed simultaneously.

9. A method of claim 3, wherein the determining step is performed by a computer-assisted device.

10. A method for predicting the response of a psoriatic arthritis patient to anti-TNFα therapy comprising:
a) determining the concentration of VEGF, prostatic acid phosphatase, and adiponectin in a blood or serum sample from said patient; and
b) comparing said concentration of VEGF in said blood or serum sample to a VEGF cutoff value, whereby if the concentration of VEGF is determined to be less than said cutoff value, the patient is predicted to be a non-responder to anti-TNFα therapy;
c) comparing the concentration of prostatic acid phosphatase in the patient's sample to a prostatic acid phosphatase cutoff value, if the serum value of VEGF is greater than or equal to the cutoff value, wherein a concentration of prostatic acid phosphatase less than a prostatic acid phosphatase cutoff value, the patient is predicted to be a responder to TNFα therapeutic, and if the PAP value greater than or equal to the PAP cutoff value, further classifying the patient using the adiponection value in the sample; wherein,
d) if the adiponectin value is less than an adiponection cutoff value the patient as predicted to be a non-responder and an adiponection value greater than or equal to a cutoff value classifies the patient as predicted to be a responder to TNFα neutralizing therapeutic.

11. The method of claim 10, wherein the sample is serum.

12. The method of claim 11 where the concentration of VEGF in serum is log transformed and the VEGF cutoff value is about 8.08.

13. The method of claim 10, wherein concentration of prostatic acid phosphatase in serum is log transformed and the prostatic acid phosphatase cutoff value is 2.29.

14. The method of claim 10, wherein concentration of adiponectin in serum is log transformed and the adiponectin cutoff value is 1.35.

15. The method of claim 10, wherein the determining step is performed simultaneously.

16. A method of claim 10, wherein the determining step is performed by a computer-assisted device.

17. A computer-based system for applying a prediction algorithm to a set of data obtained from a psoriatic arthritis patient to be treated with an anti-TNFα therapeutic and assessed using one or more clinical endpoints after treatment, comprising
a computation station for receiving and processing a patient data set in computer readable format, said computation station comprising a trained neural network for processing said patient data set and producing an output classification, wherein said trained neural network is trained with a method for preprocessing a patient data set, further comprising:
a) selecting patient biomarkers associated with PsA,
b) statistically and/or computationally testing discriminating power of the selected patient biomarkers individually in linear and/or non-linear combination for indicating the response or non-response of a patient based on a clinical endpoint,
c) applying statistical methods for the derivation of secondary inputs to the neural network that are linear or non-linear combinations of the original or transformed biomarkers,
d) selecting only those patient biomarkers or derived secondary inputs that show discriminating power; and
e) training the computer-based neural network using the preprocessed patient biomarkers or derived secondary inputs.

18. The computer-based system of claim 17, wherein the output classification is whether the patient will respond or not respond to anti-TNFα therapy and the clinical endpoints are ACR20, PsARC, or DAS28 and the biomarkers are at least two of adiponectin, prostatic acid phosphatase (PAP), MDC, SGOT, VEGF, lipoprotein A and beta-2-microgloblulin.

19. The computer-based system of claim 18, wherein in addition, the level of at least one of baseline deoxypyridinoline, S-100, hyaluronic acid, bone alkaline phosphatase alpha-1-Antitrypsin; and change from baseline to week 4 level of CRP, ENRAGE, haptoglobin, ICAM-1, IL-16, IL-18, IL-1ra, IL-8, MCP-1, MIP-1beta, MMP-3, myeloperoxidase, serum amyloid P, thyroxine binding globulin, TNFRII, and VEGF in the sample from a patient diagnosed with PsA is measured and used in the prediction.

20. A device for predicting whether a psoriatic arthritis patient to be treated with an anti-TNFα therapeutic will respond or not respond to therapy as assessed by the one or more clinical endpoints, comprising
a) a test strip comprising an antibody specific for a marker associated with a PsA patient response or non-response to anti-TNFα therapy selected from adiponectin, prostatic acid phosphatase (PAP), MCD, SGOT, VEGF, lipoprotein A and beta-2-microgloblulin, and a second antibody labeled with a detectable label;
b) detecting the signal produced by the label using a reader capable of processing the signal; and
c) processing the data obtained from the processing of the signal into a result indicative of a predetermined concentration of the marker in the sample.

21. The device of claim 20, wherein the reader is a human.

22. The device of claim 21, wherein the reader is a reflectometer.

23. A prognostic test kit for use in predicting whether a patient diagnosed with psoriatic arthritis to be treated with an anti-TNFα therapeutic will respond or not respond to therapy as assessed by the one or more clinical endpoints, comprising: a prepared substrate capable of quantifying the presence of one or more markers in a patient sample selected from adiponectin, prostatic acid phosphatase (PAP), MCD, SGOT, VEGF, lipoprotein A and beta-2-microgloblulin.
